SEQUENCE LISTING (5 sequences)

SEQ ID NO: 1 - DNA, 14 bp, Unknown
  Eukaryotic RNA polymerase III terminator sequence
nnnnnnntnnnnnn

SEQ ID NO: 2 - DNA, 17 bp, Artificial Sequence
  Synthetic Construct
aaaagcaccgactcggt

SEQ ID NO: 3 - DNA, 23 bp, Artificial Sequence
  Synthetic Construct
gttttagagctagaaatagcaag

SEQ ID NO: 4 - DNA, 21 bp, Artificial Sequence
  Synthetic Construct
catttcataaaaaggccaacc

SEQ ID NO: 5 - DNA, 22 bp, Artificial Sequence
  Synthetic Construct
cctaatgatagttcttcaatgg

(19) United States
(12) Patent Application Publication
(10) Pub. No.: US 2017/0088845 A1
(43) Pub. Date: Mar. 30, 2017

(54) VECTORS AND METHODS FOR FUNGAL GENOME ENGINEERING BY CRISPR-CAS9

(71) Applicants: The Regents of the University of California, Oakland, CA (US); BP Corporation North America Inc., Houston, TX (US)

(72) Inventors: Owen RYAN, San Francisco, CA (US); James H. DOUDNA CATE, Berkeley, CA (US); David Neal NUNN, JR., Carlsbad, CA (US)

(73) Assignees: The Regents of the University of California, Oakland, CA (US); BP Corporation North America Inc., Houston, TX (US)

(21) Appl. No.: 15/125,948

(22) PCT Filed: Mar. 13, 2015
(86) PCT No.: PCT/US15/20377

§ 371 Date: Sep. 13, 2016

Related U.S. Application Data
(60) Provisional application No. 61/953,600, filed on Mar. 14, 2014.

(51) Int. Cl. C12N 15/81

(52) U.S. Cl. CPC C12N 15/81

(57) ABSTRACT

The present disclosure provides expression vectors containing a nucleic acid encoding an RNA polymerase III promoter, a ribozyme, a CRISPR-Cas9 single guide RNA, and an RNA polymerase III terminator, where the ribozyme is 5′ to the CRISPR-Cas9 single guide RNA, as well as ribonucleic acids encoded thereby. Further provided are fungal cells containing an expression vector described herein, as well as methods of fungal genome engineering through use of an expression vector described herein.

### CROSS-REFERENCE TO RELATED APPLICATIONS

This application claims the benefit of U.S. Provisional Application No. 61/953,600, filed Mar. 14, 2014, which is hereby incorporated by reference in its entirety.

### FIELD OF THE INVENTION

The present disclosure relates to expression vectors containing a nucleic acid encoding an RNA polymerase III promoter, a ribozyme, a CRISPR-Cas9 single guide RNA, and an RNA polymerase III terminator, where the ribozyme is 5′ to the CRISPR-Cas9 single guide RNA, as well as ribonucleic acids encoded thereby. These expression vectors and ribonucleic acids may find use, for example, in fungal cells and in methods of fungal genome engineering.

### BACKGROUND

Renewable energy is of global importance due to the effects of global warming, the reduction of natural resources, and the extreme fluctuations in the cost of oil. Biofuel production by cellulosic fermentation has the potential for creating a renewable and greenhouse gas reducing form of transportation fuel.

A general method of cellulosic biofuel production may involve converting solar energy into plant cell wall biomass, hydrolytic saccharification of plant cell wall polysaccharides, and fermentation of monosaccharides and disaccharides by yeast into ethanol. For industrial fermentation, the yeast species typically used is baker's yeast, Saccharomyces cerevisiae. However, industrial S. cerevisiae strains are more stress tolerant and produce much higher yields of ethanol than the model laboratory strain, S288c. S288c optimally ferments glucose into ethanol at ˜30° C. and is limited in its natural inability to ferment xylose or cellobiose, making it unsuitable for industrial scale biofuel fermentations.

Unfortunately, the genetic basis of many of the desired industrial yeast phenotypes remains unknown. This is because industrial yeast strains tend to be polyploidy, and standard genetic tools based on the integration of linear DNA by homologous recombination (HR) are not efficient enough for the creation of loss-of-function alleles in polyploids or modifiying multiple loci simultaneously for synthetic biology applications. Further, current technologies allow for only a very limited number of genome integrations because each integration must be linked to a dominant selectable marker, so creating homozygous mutants requires the use of two or more markers for any single locus.

The bacterial type II CRISPR-Cas9 programmable RNA genome editing method has recently received a great deal of interest in the field of genome engineering. The co-expression of a single Cas9 protein isolated from Streptococcus pyogenes with a chimeric single guide RNA (sgRNA) can precisely create double stranded breaks (DSBs) in a genome (Jinek, M., et al. (2012) Science 337(6096):816-21; Mali, P., et al. (2013) Science 339(6121):823-6). The Cas9 protein is directed to a precise DNA sequence in the genome by a twenty nucleotide target sequence present in the sgRNA, which guides the Cas9 protein to create the DSB. The presence of a DSB in genomic DNA increases the rate of HR by over a thousand-fold (Storici, F., et al. (2003) Proc. Natl. Acad. Sci. USA 100(25):14994-9).

Cas-mediated genome editing has been disclosed for S. cerevisiae haploid strains (DiCarlo, J. E., et al. (2013) Nucleic Acids Res. 41(7):4336-43). However, the efficiency of this system is far below that required for high-throughput screening or systems biology applications using polyploid yeast cells (e.g., industrial yeast strains). Therefore, a need exists for an improved genome editing method that works efficiently with polyploid yeast and multiple genomic loci for multiplexed genome editing.

### BRIEF SUMMARY

Certain aspects of the present disclosure relate to expression vectors containing nucleic acid encoding an RNA polymerase III promoter, a ribozyme, CRISPR-Cas 9 single guide RNA, and an RNA Polymerase III terminator, where the ribozyme is 5′ to the CRISPR-Cas9 single guide RNA. In some embodiments, the vector further contains nucleic acid encoding a Cas9 protein. In some embodiments that may be combined with any of the preceding embodiments, the CRISPR-Cas9 single guide RNA contains a 20 nucleotide target sequence and a sgRNA (+85) tail. In some embodiments, the RNA polymerase III promoter is a tRNA. In some embodiments, the tRNA is a tyrosine tRNA. In some embodiments, the RNA polymerase III promoter is a non-tRNA promoter. In some embodiments, the non-tRNA promoter is SNR52. In some embodiments that may be combined with any of the preceding embodiments, the ribozyme is self-cleaving. In some embodiments that may be combined with any of the preceding embodiments, the ribozyme is active between 30° C. and 37° C. In some embodiments that may be combined with any of the preceding embodiments, the ribozyme is a hepatitis delta ribozyme. In some embodiments that may be combined with any of the preceding embodiments, the vector contains more than one CRISPR-Cas 9 single guide RNA.

Further aspects of the present disclosure relate to ribonucleic acids encoded by the expression vector of any of the preceding embodiments.

Yet further aspects of the present disclosure relate to fungal cells containing an expression vector of any of the preceding embodiments. In some embodiments, the cell is an industrial strain. In some embodiments, the cell is polyploid. In some embodiments, the cell is diploid. In some embodiments, the cell is a filamentous fungal cell. In some embodiments, the cell is a yeast cell. In some embodiments, the yeast cell is Saccharomyces cerevisiae, Kluyveromyces marxianus, or Issatchenkia orientalis.

Yet further aspects of the present disclosure relate to methods for engineering a fungal genome, including introducing an expression vector of any of the preceding embodiments and an expression vector encoding a Cas9 protein into a fungal cell, and culturing the cell under conditions suitable for expression. Yet further aspects of the present disclosure relate to methods for engineering a fungal genome, including introducing an expression vector containing nucleic acid encoding an RNA polymerase III promoter, a ribozyme, CRISPR-Cas 9 single guide RNA, and an RNA Polymerase III terminator, and a Cas9 protein, where the ribozyme is 5′ to the CRISPR-Cas9 single guide RNA, into a fungal cell, and culturing the cell under conditions suitable for expression. In some embodiments, the methods further include introducing a nucleic acid encoding a gene of interest. In some embodiments, the gene of interest is a cellodextrin transporter. In some embodiments that may be combined with any of the preceding embodiments, the gene of interest is encoded by more than one polynucleotide. In some embodiments that may be combined with any of the preceding embodiments, the gene of interest is generated by error-prone PCR.

It is to be understood that one, some, or all of the properties of the various embodiments described herein may be combined to form other embodiments of the present disclosure. These and other aspects of the present disclosure will become apparent to one of skill in the art.

### DESCRIPTION OF THE FIGURES

The patent or application file contains at least one drawing executed in color. Copies of this patent or patent application publication with color drawings will be provided by the office upon request and payment of the necessary fee.

FIG. 1 shows an exemplary embodiment of a tRNA promoter-driven ribozyme-sgRNA system, including an expression construct (A), mature RNA (A), and folded RNA after promoter removal, with various features labeled (B).

FIG. 2 shows the engineering of a Cas9 protein functional in yeast cells. (A) Schematic representation of a GFP-tagged Cas9 protein. (B) Corresponding bright field and GFP fluorescence microscopic images showing that GFP-tagged Cas9 localizes to the nucleus in yeast cells.

FIG. 3 shows that the presence of a 5′ ribozyme increases sgRNA abundance as expressed by an RNA Pol II (A) or RNA Pol III (B) promoter. RNA abundance is measured by qRT-PCR and expressed as fold expression of ribozyme (+) RNA compared to ribozyme (−) RNA.

FIG. 4 illustrates an overview of a CRISPR-Cas9 genome editing system.

FIG. 5 shows how a linear barcode DNA (A) may be used to facilitate Cas-mediated genome editing, with various steps illustrated (B). Barcode features, including flanking 50 bp homology regions, stop codon, forward and reverse primer binding sites (Pr. F and Pr. R) and 20-mer barcode, are labeled.

FIG. 6 illustrates an overview of a yeast screening model for examining Cas-mediated genome editing.

FIG. 7 shows several assays involved in a yeast screening model. Plating of transformants on selective media to select for transformation (1), plating on selective media to select for mutation of a selectable locus (2), PCR to detect barcode in genomic DNA (3), and sequencing to confirm barcode integration (4) are shown.

FIG. 8 shows that the efficiency of duplex targeting of URA3 and LYP1 simultaneously in diploid S288C yeast cells is enhanced by the presence of a 5′ ribozyme. When both sgRNAs contain a 5′ ribozyme, targeting efficiency is 43%; when both sgRNAs lack a 5′ ribozyme, targeting efficiency is 3.5%.

FIG. 9 shows the targeting efficiency of Cas-mediated genome editing of the URA3 locus in diploid yeast cells, comparing different RNA Pol III promoters for sgRNA expression as labeled (note that tRNA promoters are labeled by their cognate amino acid).

FIG. 10 shows the targeting efficiency of Cas-mediated genome editing at different genomic loci (as labeled). Note that initial targeting of LEU2 was not efficient (LEU2), but using a different LEU2 targeting sequence (LEU2-2) was able to restore efficient targeting.

FIG. 11 shows the quantification of mutations detected near PAM sites in yeast strains targeted at the URA3 or LYP1 loci, as detected by whole genome sequencing.

FIG. 12 shows the targeting efficiency of Cas-mediated genome editing of the URA3 locus in S288C diploid and ATCC4124 polyploid yeast cells. Different promoters for sgRNA expression were examined (SNR52, tRNA^{Tyr}, tRNA^{Pro}, and tRNA^{Phe} promoters, as indicated).

FIG. 13 illustrates an exemplary Cas-mediated genome editing process for integrating a functional nourseothricin-resistance (Nat^{R}) gene cassette (including the TEF1 promoter and terminator of Ashbya gosypii) (A). (B) Targeting efficiency using the Nat^{R} gene cassette in lab (diploid S288C) and industrial (ATCC4124) yeast strains, using different sgRNA promoters as indicated.

FIG. 14 shows the efficiency of assembling the NatR drug cassette in haploid lab yeast (S288C 1n), diploid lab yeast (S288C 2n), and two isolates of industrial yeasts (JAY270 and ATCC4124).

FIG. 15 illustrates Cas-mediated multiplex genome editing (A). (B) Targeting efficiency (expressed as a percentage) using haploid or diploid S288C cells (as indicated) and targeting 1, 2, or 3 genetic loci.

FIG. 16 shows an exemplary overview of a screen for generating improved cellobiose utilizing strains using Cas-mediated genome editing.

FIG. 17 shows the growth of an improved cellobiose utilizing strain generated by Cas-mediated genome editing. Optical densities of cultures grown in cellobiose medium are plotted over time. Data are provided for S288C without cdt-1, a positive control of S288C with wild-type cdt-1, and S288C with a cdt-1 mutant (G626A), as labeled.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

The present disclosure relates generally to expression vectors containing a nucleic acid encoding an RNA polymerase III promoter, a ribozyme, a CRISPR-Cas9 single guide RNA, and an RNA polymerase III terminator, where the ribozyme is 5′ to the CRISPR-Cas9 single guide RNA, as well as ribonucleic acids encoded thereby. Further embodiments relate generally to fungal cells containing an expression vector described herein, as well as methods of fungal genome engineering through use of an expression vector described herein.

In particular, the present disclosure is based, at least in part, on the surprising discovery that the presence of a ribozyme in a CRISPR-Cas9 single guide RNA increases CRISPR-Cas9 single guide RNA abundance and/or the efficiency of genome engineering by CRISPR-Cas9. Moreover, the use of a CRISPR-Cas9 single guide RNA containing a ribozyme increases the targeting efficiency of genome engineering in polyploid fungal strains, industrial fungal strains, and in multiplex applications wherein multiple genomic loci are targeted simultaneously.

Accordingly, the present disclosure provides expression vectors containing a nucleic acid encoding an RNA polymerase III promoter, a ribozyme, a CRISPR-Cas9 single guide RNA, and an RNA polymerase III terminator, where the ribozyme is 5′ to the CRISPR-Cas9 single guide RNA, as well as ribonucleic acids encoded thereby. Further provided are fungal cells containing an expression vector described herein, as well as methods of fungal genome engineering through use of an expression vector described herein. These expression vectors and methods allow for fungal genome engineering with enhanced targeting efficiency.

### CRISPR-Cas9 Expression Vectors and Ribonucleic Acids

Certain aspects of the present disclosure relate to an expression vector containing nucleic acid encoding an RNA polymerase III promoter, a ribozyme, a CRISPR-Cas 9 single guide RNA, and an RNA Polymerase III terminator, where the ribozyme is 5′ to the CRISPR-Cas9 single guide RNA.

CRISPR-Cas9 and CRISPR-Cas9 Single Guide RNA

As used herein, “CRISPR-Cas9” refers to a two component ribonucleoprotein complex with guide RNA and a Cas9 endonuclease. CRISPR refers to the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner (Jinek, M., et al. (2012) Science 337(6096):816-21). In type II systems, guide RNA interacts with Cas9 and directs the nuclease activity of Cas9 to target DNA sequences complementary to those present in the guide RNA. Guide RNA base pairs with complementary sequence in target DNA. Cas9 nuclease activity then generates a double-stranded break in the target DNA.

In bacteria, Cas9 polypeptides bind to two different guide RNAs acting in concert: a CRISPR RNA (crRNA) and a trans-activating crRNA (tracrRNA). The crRNA and tracrRNA ribonucleotides base pair and form a structure required for the Cas9-mediated cleavage of target DNA. However, it has recently been demonstrated that a single guide RNA (sgRNA) may be engineered to form the crRNA:tracrRNA structure and direct Cas9-mediated cleavage of target DNA (Jinek, M., et al. (2012) Science 337(6096):816-21). Moreover, since the specificity of Cas9 nuclease activity is determined by the guide RNA, the CRISPR-Cas9 system has been explored as a tool to direct double-stranded DNA breaks in heterologous cells, enabling customizable genome editing (Mali, P., et al. (2013) Science 339(6121):823-6).

As used herein, “CRISPR-Cas9 single guide RNA” (the terms “single guide RNA” and “sgRNA” may be used interchangeably herein) refers to a single RNA species capable of directing Cas9-mediated cleavage of target DNA. In some embodiments, a single guide RNA may contain the sequences necessary for Cas9 nuclease activity and a target sequence complementary to a target DNA of interest.

As used herein, an sgRNA target sequence refers to the nucleotide sequence of an sgRNA that binds to a target DNA sequence and directs Cas9 nuclease activity to that DNA locus. In some embodiments, the sgRNA target sequence is complementary to the target DNA sequence. As described herein, the target sequence of a single guide RNA may be customized, allowing the targeting of Cas9 activity to a target DNA of interest. For a more detailed description of how sgRNA sequence may be customized for different target sequences, see Mali, P., et al. (2013) Science 339(6121):823-6.

Any desired target DNA sequence of interest may be targeted by an sgRNA target sequence. Without wishing to be bound to theory, it is thought that the only requirement for a target DNA sequence is the presence of a protospacer-adjacent motif (PAM) adjacent to the sequence complementary to the sgRNA target sequence (Mali, P., et al. (2013) Science 339(6121):823-6). Different Cas9 complexes are known to have different PAM motifs. For example, Cas9 from Streptococcus pyogenes has a GG dinucleotide PAM motif. For further examples, the PAM motif of N. meningitidis Cas9 is GATT, the PAM motif of S. thermophilus Cas9 is AGAA, and the PAM motif of T. denticola Cas9 is AAAAC.

In some embodiments, a single guide RNA contains a 20 nucleotide target sequence. Any length of target sequence that permits CRISPR-Cas9 specific nuclease activity may be used in a single guide RNA.

In some embodiments, a single guide RNA may contain an sgRNA (+85) tail. As used herein, an “sgRNA (+85) tail” may refer to an 85 base pair sequence contained in an sgRNA polynucleotide that facilitates CRISPR-Cas9 activity but does not determine the target sequence of the CRISPR-Cas9 complex. For example, an sgRNA (+85) tail has been demonstrated to act as a tracrRNA and promote CRISPR-Cas9 activity (Hsu, P. D., et al. (2013) Nat. Biotech. 31:827-32). In some embodiments, a single guide RNA may contain an sgRNA (+67) tail. Any sgRNA (+85) tail sequence known in the art may be used.

In some embodiments, the vector further contains nucleic acid encoding a Cas9 protein. As used herein, a “Cas9” polypeptide is a polypeptide that functions as a nuclease when complexed to a guide RNA, e.g., an sgRNA. The Cas9 (CRISPR-associated 9, also known as Csn1) family of polypeptides, when bound to a crRNA:tracrRNA guide or single guide RNA, are able to cleave target DNA at a sequence complementary to the sgRNA target sequence and adjacent to a PAM motif as described above. Unlike other Cas polypeptides, Cas9 polypeptides are characteristic of type II CRISPR-Cas systems (for a description of Cas proteins of different CRISPR-Cas systems, see Makarova, K. S., et al. (2011) Nat. Rev. Microbiol. 9(6):467-77). As used herein, “Cas9” may refer to the ribonucleoprotein complex with an sgRNA or the polypeptide component of the complex, unless specified.

In some embodiments, a Cas9 polypeptide refers to a Cas9 polypeptide derived from Streptococcus pyogenes, e.g., a polypeptide having the sequence of the Swiss-Prot accession Q99ZW2. In some embodiments, a Cas9 polypeptide refers to a Cas9 polypeptide derived from Streptococcus thermophilus, e.g., a polypeptide having the sequence of the Swiss-Prot accession G3ECR1. In some embodiments, a Cas9 polypeptide refers to a Cas9 polypeptide derived from a bacterial species within the genus Streptococcus. In some embodiments, a Cas9 polypeptide refers to a Cas9 polypeptide derived from a bacterial species within the genus Neisseria (e.g., GenBank accession number YP_003082577). In some embodiments, a Cas9 polypeptide refers to a Cas9 polypeptide derived from a bacterial species within the genus Treponema (e.g., GenBank accession number EMB41078). In some embodiments, a Cas9 polypeptide refers to a polypeptide with Cas9 activity as described above derived from a bacterial or archaeal species. Methods of identifying a Cas9 protein are known in the art. For example, a putative Cas9 protein may be complexed with crRNA and tracrRNA or sgRNA and incubated with DNA bearing a target DNA sequence and a PAM motif, as described in Jinek, M., et al. (2012) Science 337(6096):816-21.

Cas9 polypeptides cleave target DNA, directed by guide RNA, through nuclease activity. Two nuclease domains, a RuvC-like domain and an HNH (a.k.a. McrA-like) domain, catalyze the nuclease activity. For a double-stranded DNA substrate, the HNH domain is thought to cleave the strand complementary to the sgRNA target sequence, and the RuvC-like domain is thought to cleave the non-complementary strand. These cleavages produce a double-stranded break at a DNA target site with an adjacent PAM motif.

Ribozymes

As used herein, a “ribozyme” refers to an RNA molecule (which may be non-coding) that possesses a catalytic or enzymatic activity. Ribozymes may be identified, for example, by possessing a measurable enzymatic activity, e.g., self-cleavage, or they may be identified by a prediction of secondary structure based upon their RNA sequence. Ribozyme activity is known to be influenced by ribozyme secondary structure and/or tertiary folding. As such, ribozymes with common activity may not share the same RNA sequence, but rather they may share a common pattern of base pairing that yields a common secondary structure. Tools for predicting RNA secondary structure are known in the art (see, e.g., the web-based fold predictor available at rna.tib.univie.ac.at/cgi-bin/RNAfold.cgi). For a more detailed description of ribozymes, see Serganov, A. and Patel, D. J. (2007) Nat. Rev. Genet. 8:776-90.

In some embodiments, the ribozyme is self-cleaving. As used herein, a “self-cleaving ribozyme” refers to a ribozyme that is able to cleave itself into two separate ribonucleotides. For example, a self-cleaving ribozyme may catalyze the reaction characterized by a 2′-hydroxyl attack on the ribonucleic acid, yielding free 5′-OH and 2′,3′-cyclic phosphate termini. Any self-cleaving ribozyme known in the art may be used. Examples of self-cleaving ribozymes may include, without limitation, hepatitis delta virus (HDV), hammerhead, hairpin, and Varkud satellite (VS) ribozymes.

In some embodiments, a ribozyme is 5′ to a CRISPR-Cas9 single guide RNA. In some embodiments, a ribozyme is encoded by the same nucleic acid as a CRISPR-Cas9 single guide RNA. As used herein, the directions 5′ and 3′ refer to the asymmetric ends of a polynucleotide molecule. 5′ refers to the end with a terminal phosphate. 3′ refers to the end with the terminal hydroxyl.

In some embodiments, a ribozyme has self-cleavage activity against sequences 5′ to its own sequence, e.g., as with a hepatitis delta ribozyme. In some embodiments, a self-cleaving ribozyme may be used to separate a single guide RNA from another sequence, e.g., a tRNA sequence, immediately 5′ to the single guide RNA sequence.

In some embodiments, a ribozyme is active between about 30° C. and about 37° C. Many types of cells preferentially proliferate, grow, and/or produce a product (e.g., a compound or polypeptide) between about 30° C. and about 37° C. For example, many yeast strains are typically grown between about 30° C. and about 37° C. In some embodiments, a ribozyme is used that is enzymatically active in the same temperature range as the preferred temperature range for growth of the cell in which it is expressed.

In some embodiments, the self-cleaving ribozyme is a hepatitis delta ribozyme (the terms “hepatitis delta virus” ribozyme and HDV ribozyme may be used interchangeably herein). As used herein, hepatitis delta ribozyme refers to ribozyme derived from HDV that catalyzes self-cleavage of sequence immediately 5′ to its own. HDV ribozyme secondary structure is characterized by 5 helical segments connected by a double pseudoknot. Self-cleaving ribozymes derived from viruses like HDV may participate in rolling-circle replication of viral RNA. Sequences of hepatitis delta ribozymes are known in the art (Been, M. D. and Wickham, G. S. (1997) Eur. J. Biochem. 247:741-53; Chadalavada, D. M., et al. (2007) RNA 13(12):2189-2201). Since ribozyme activity is influenced by secondary structure, hepatitis delta ribozymes may diverge in sequence but still retain common secondary structure, tertiary folding, and/or activity.

RNA Polymerase III Promoters and Terminators

As used herein, an “RNA polymerase III promoter” (RNA Pol III or Pol III promoter) refers to a nucleotide sequence that directs the transcription of RNA by RNA polymerase III. RNA polymerase III promoters may include a full-length promoter or a fragment thereof sufficient to drive transcription by RNA polymerase III. For a more detailed description of RNA polymerase III promoter types, structural features, and interactions with RNA polymerase III, as well as suitable RNA polymerase III promoters, see Schramm, L. and Hernandez, N. (2002) Genes Dev. 16:2593-620.

As used herein, a “promoter” may refer to any nucleic acid sequence that regulates the initiation of transcription for a particular polypeptide-encoding nucleic acid under its control. A promoter minimally includes the genetic elements necessary for the initiation of transcription (e.g., RNA polymerase III-mediated transcription), and may further include one or more genetic elements that serve to specify the prerequisite conditions for transcriptional initiation. A promoter may be encoded by the endogenous genome of a host cell, or it may be introduced as part of a recombinantly engineered polynucleotide. A promoter sequence may be taken from one host species and used to drive expression of a gene in a host cell of a different species. A promoter sequence may also be artificially designed for a particular mode of expression in a particular species, through random mutation or rational design. In recombinant engineering applications, specific promoters are used to express a recombinant gene under a desired set of physiological or temporal conditions or to modulate the amount of expression of a recombinant nucleic acid.

Many RNA polymerase III promoters are known in the art. In some embodiments, an RNA polymerase III promoter may be a tRNA. tRNA promoters are known to be intragenic and class II RNA polymerase III promoters. For example, tRNA sequences may contain A- and B-boxes, which are bound by TFIIIC as a step in RNA polymerase III transcriptional initiation. In some embodiments, the tRNA may be a tyrosine tRNA. Any tRNA corresponding to any amino acid may be used as a promoter to direct RNA polymerase III-mediated gene expression.

In some embodiments, an RNA polymerase III promoter may be a non-tRNA promoter. Examples of non-tRNA RNA polymerase III promoters may include, without limitation, promoters for 5S RNA, U6 snRNA, 7SK, RNase P, the RNA component of the Signal Recognition Particle, and snoRNAs. Examples of non-tRNA promoters may include class I and class III RNA polymerase III promoters. For a more detailed description of non-tRNA promoters, see Orioli, A., et al. (2012) Gene 493(2):185-94.

In some embodiments, the non-tRNA promoter may be the SNR52 promoter. As used herein, SNR52 refers to a C/D box small nucleolar RNA (snoRNA) involved in methylation of rRNA. As used herein, an SNR52 promoter may refer to a full-length promoter sequence, or a fragment thereof, linked to an SNR52 gene that is sufficient to drive transcription mediated by RNA polymerase III. Examples of SNR52 genes may include, e.g., S. cerevisiae SNR52.

As used herein, an “RNA polymerase III terminator” refers to any nucleotide sequence that is sufficient to terminate a transcript transcribed by RNA polymerase III. As used herein, and unless specified, an RNA polymerase III terminator may refer to the transcribed RNA sequence itself or the DNA sequence encoding it. Examples of RNA polymerase III terminators may include, without limitation, a string of uridine nucleotides of at least 5-6 bases in length (for more information on RNA polymerase III terminators, see Marck, C., et al. (2006) Nucleic Acids Res 34(6):1816-35). In some embodiments, the RNA polymerase III terminator is UUUUUUUTUUUUUU.

Expression Vectors

As used herein, an “expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside a given host cell. For example, an expression vector may contain sequences encoding an RNA polymerase III promoter, a self-cleaving ribozyme, a single guide RNA, an RNA polymerase III terminator, and/or a Cas9 protein. As used herein, a “host cell” refers to a cell that contains an expression vector.

Many suitable expression vectors and features thereof are known in the art; for example, various vectors and techniques are illustrated in Current Protocols in Molecular FExpression vectors may contain, without limitation, a centromeric (CEN) sequence, an autonomous replication sequence (ARS), a promoter, an origin of replication, and a marker gene (e.g., auxotrophic, antibiotic, or other selectable markers). Examples of expression vectors may include plasmids, yeast artificial chromosomes, 2 μm plasmids, yeast integrative plasmids, yeast replicative plasmids, shuttle vectors, and episomal plasmids.

Methods for transforming a host cell with an expression vector may differ depending upon the species of the desired host cell. For example, yeast cells may be transformed by lithium acetate treatment (which may further include carrier DNA and PEG treatment) or electroporation. These methods are included for illustrative purposes and are in no way intended to be limiting or comprehensive. Routine experimentation through means well known in the art may be used to determine whether a particular expression vector or transformation method is suited for a given host cell. Furthermore, reagents and vectors suitable for many different host microorganisms are commercially available and/or well known in the art.

Ribonucleic Acids

Further aspects of the present disclosure relate to a ribonucleic acid encoded by an expression vector of the present disclosure. Unless specified, references to an expression vector or any sequence thereof may generically refer to the DNA of the expression vector or any RNA encoded thereby. In some embodiments, a ribonucleic acid encoded by an expression vector may be coding, i.e., it encodes a transcript that is translated into a polypeptide. For example, a coding ribonucleic acid may include an RNA encoding a Cas9 protein. In some embodiments, a ribonucleic acid encoded by an expression vector may be non-coding, i.e., it encodes a transcript that is not translated into a polypeptide. For example, a non-coding ribonucleic acid may include a ribozyme, RNA polymerase III promoter, RNA polymerase III terminator, or single guide RNA.

### Fungal Cells

Certain aspects of the present disclosure relate to a fungal cell containing an expression vector of the present disclosure.

As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi.

In some embodiments, the fungal cell is a yeast cell. As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum).

In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

Expression vectors and techniques suitable for the maintenance, construction, propagation, and transformation thereof in a variety of fungal cells are known in the art. Further details of expression vectors and techniques may be found in Yeast Protocols, 2^{nd} edition, Xiao, W., ed. (Humana Press, New York, 2007) and Buckholz, R. G. and Gleeson, M. A. (1991) Biotechnology (NY) 9(11):1067-72.

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains may include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest. Without wishing to be bound to theory, it is thought that the abundance of sgRNA may more often be a rate-limiting component in genome engineering of polyploid cells than in haploid cells, and thus the methods described herein may be advantageous for these applications.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest.

In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

As used herein, a “host cell” refers to a cell transformed or transfected with an expression vector or other nucleic acid. In some embodiments, a host cell is able to promote an expression vector's replication, maintenance, and/or expression of a nucleic acid.

The expression vectors and methods described herein may further be adapted to use a host cell that is not a fungal cell. Examples of other suitable host cells may include, without limitation, human cells, mammalian cells, bacterial cells, plant cells, insect cells, and animal cells.

### Methods for Engineering a Yeast Genome

### Cas9-Mediated Genome Engineering

Certain aspects of the present disclosure relate to methods for engineering a fungal genome. In some embodiments, a fungal genome is engineered by introducing an expression vector containing nucleic acid encoding an RNA polymerase III promoter, a self-cleaving ribozyme, a CRISPR-Cas 9 single guide RNA, and an RNA Polymerase III terminator and an expression vector encoding a Cas9 protein into a fungal cell; and culturing the cell under conditions where the vectors are expressed. In some embodiments, the expression vector contains a nucleic acid encoding an RNA polymerase III promoter, a self-cleaving ribozyme, a CRISPR-Cas 9 single guide RNA, and an RNA Polymerase III terminator and a nucleic acid encoding a Cas9 protein.

As used herein, “genome engineering” (the term “genome editing” is used interchangeably herein) refers to the modification of a genome through targeted mutagenesis (e.g., through use of a Cas9 protein and an sgRNA containing a target sequence). In some embodiments, the RNA containing a self-cleaving ribozyme and single guide RNA (which also may include an RNA polymerase III promoter and terminator) is expressed in a host cell that also expresses a Cas9 protein. The single guide RNA is able to complex with the Cas9 protein to generate a functional CRISPR-Cas9 complex. When the single guide RNA contains a target sequence that binds a DNA target sequence in the genome of the cell in which the CRISPR-Cas9 complex is expressed, the CRISPR-Cas9 complex may modify the host cell genome, e.g., by inducing a double stranded break at a DNA target sequence. Constructing a single guide RNA with a target sequence complementary to a DNA target sequence (adjacent to a PAM motif recognized by the Cas9 protein expressed) in a genomic locus of interest may enable the direction of nuclease activity to the genomic locus of interest. For a more detailed description, see Mali, P., et al. (2013) Science 339(6121):823-6.

In some embodiments, the genomic DNA target sequence is adjacent to a PAM motif. As described earlier, a PAM motif is recognized by a CRISPR-Cas9 complex, and the specific sequence of the PAM motif is determined by the type of Cas9 protein.

The nuclease activity of a CRISPR-Cas9 complex results in cleavage at a DNA target sequence. In some embodiments, a CRISPR-Cas9 complex induces a double-stranded break at a DNA target sequence. Upon detection of a double-stranded break at a genomic locus, cells are known to initiate specific repair pathways. These pathways may be advantageously used in genome engineering to create a mutation, insert DNA sequence, or delete DNA sequence at the site of the double-stranded break.

One mechanism for double-stranded break repair is by homologous recombination (HR) (Jasin, M. and Rothstein, R. (2013) Cold Spring Harb. Perspect. Biol. 5(11):a012740). During HR, a double-stranded break is repaired using sequences with homology to the DNA flanking the break as a template. In genome engineering, a linear DNA polynucleotide, flanked with sequences (e.g., of 50 base pairs or more) homologous to a genomic locus targeted by a double-stranded break, is introduced when the double-stranded break is induced. The host cell's endogenous double-stranded break repair pathway then uses the linear DNA as a template, resulting in the genomic integration of the linear DNA between the flanking homologous sequences. In some embodiments, this approach is used to introduce a DNA sequence at a genomic locus. In some embodiments, this approach is used to delete a DNA sequence present at a genomic locus.

Another mechanism for double-stranded break repair is non-homologous end-joining. This mechanism does not repair a double-stranded break through HR, but rather through ligating the break ends directly without a homologous template, or with a microhomology sequence.

The process of double-stranded break repair may repair the break cleanly, i.e., without altering the starting sequence. The process of double-stranded break repair may alternatively induce a mutation through an error in repair. In some embodiments, genome engineering is used to create a DNA deletion of one or more base pairs. In some embodiments, genome engineering is used to create a DNA insertion of one or more base pairs. In some embodiments, genome engineering is used to create a mutation (e.g., point mutation or single nucleotide polymorphism, or SNP).

In some embodiments, genome engineering is carried out at more than one genomic locus simultaneously (i.e., multiplex genome engineering). In some embodiments, an expression vector is used than contains more than one single guide RNA. In some embodiments, expression of more than one single guide RNA results in more than one species of CRISPR-Cas9 complex present in a host cell. If CRISPR-Cas9 complexes contain single guide RNA with more than one target sequences, then more than one DNA target sequences may be modified by genome engineering. Without wishing to be bound to theory, it is thought that the abundance of sgRNA may more often be a rate-limiting component in multiplex genome engineering than with single genome engineering applications, and thus the methods described herein may be advantageous for these applications.

In some embodiments, methods for engineering a fungal genome may include introducing a nucleic acid encoding a gene of interest. As described above, genome engineering may be used to insert a DNA sequence (e.g., a gene) into a genomic locus. In some embodiments, the nucleic acid encoding a gene of interest is encoded by an expression vector. In some embodiments, the nucleic acid encoding a gene of interest is encoded by DNA sequence separate from the expression vector. For example, and without limitation, the nucleic acid encoding a gene of interest may be a linear DNA polynucleotide that is co-transformed with an expression vector (e.g., a linear DNA barcode).

Any gene of interest may be introduced. A gene of interest may include an RNA molecule with a desired activity, a DNA molecule with a desired activity (e.g., encoding a polypeptide, representing a detectable marker, etc.), or a nucleic acid encoding a polypeptide of interest. Polypeptides of interest may include enzymes with a desired biochemical activity, a polypeptide product of interest, or a polypeptide with a desired regulatory activity. A gene of interest may be used to replace a gene in the genome with a copy bearing an altered sequence, e.g., to replace a mutation present in the genome, or to add a mutation in a genome. Examples of genes of interest may include, without limitation, genes involved in a xylose utilization pathway and genes involved in a cellobiose utilization pathway. Examples of genes involved in xylose utilization pathways and cellobiose utilization pathways may include, without limitation, those described in Ha, S. J., et al. (2011) Proc. Natl. Acad. Sci. USA 108(2):504-9 and Galazka, J. M., et al. (2010) Science 330(6000):84-6.

In some embodiments, the gene of interest is a cellodextrin transporter. As used herein, a “cellodextrin transporter” refers to a polypeptide with the enzymatic activity of transporting cellodextrin. Transporting cellodextrin may refer to directing the movement of cellobiose into, out of, or within a cell. Any polypeptide known or predicted to have the biological activity representing by GO term GO:0019533 may be a cellodextrin transporter as described herein. Examples of cellodextrin transporters may include without limitation N. crassa CDT-1 and CDT-2 as described in Galazka, J. M., et al. (2010) Science 330(6000):84-6. Methods for identifying cellodextrin transporters are known in the art and may include transforming a non-cellobiose-utilizing yeast cell with DNA encoding a potential cellodextrin transporter, growing the cell in a medium with cellobiose as the sole carbon source, and measuring cell growth over time (e.g., by optical density).

As used herein, “cellodextrin” refers to a glucose polymer made of glucose monomers linked by β-1,4 glycosidic bonds. Examples of cellodextrin may include, without limitation, cellobiose, cellotriose, cellotetraose, cellopentaose, and cellohexaose.

In some embodiments, a gene of interest is encoded by more than one polynucleotide. For example, as demonstrated in Example 5 of the present disclosure, genome engineering may be used to introduce a gene of interest encoded by multiple, separate polynucleotides (e.g., multiple, separate, linear DNA molecules with overlapping sequence, e.g., of 50 base pairs or more). In some embodiments, a gene of interest is encoded by one polynucleotide.

### Generation and Testing of Gene Mutants Through Cas9-Mediated Genome Engineering

The expression vectors and methods described herein allow rapid and efficient integration of a gene of interest into a host cell genome. In some embodiments, these expression vectors and methods may be used to test the function of multiple genes of interest upon integration into a host cell genome. For example, a series of genes of interest, representing a plurality of variants or mutants (e.g., a library), may be integrated into the genomes of a plurality of host cells, such that each host cell integrates a different variant or mutant into its genome. Because a gene of interest is rapidly integrated into a host cell (in contrast to, e.g., a transformation, which requires more lengthy growth and selection steps), these expression vectors and methods may find use in rapidly screening a library of gene variants for a desired phenotype, e.g., utilization of xylose or cellobiose.

In some embodiments, a gene of interest is generated by error-prone PCR. Error-prone PCR refers to a technique known in the art for generating and amplifying mutated DNA sequences. Generally, this technique is similar to traditional PCR, except that it is carried out using a DNA polymerase that lacks proof-reading ability and hence has a higher error rate than a DNA polymerase with proof-reading ability. This technique may be used, e.g., to generate a library of variant or mutated copies of a DNA template (e.g., a gene of interest). For a more detailed description of error-prone PCR, see McCullum, E. O., et al. (2010) Methods Mol. Biol. 634:103-9.

### Cell Culturing

Certain aspects of the present disclosure relate to methods of culturing a cell. As defined herein, “culturing” a cell refers to introducing an appropriate culture medium, under appropriate conditions, to promote the growth of a cell. Methods of culturing various types of cells are known in the art. Culturing may be performed using a liquid or solid growth medium. Culturing may be performed under aerobic or anaerobic conditions where aerobic, anoxic, or anaerobic conditions are preferred based on the requirements of the microorganism and desired metabolic state of the microorganism. In addition to oxygen levels, other important conditions may include, without limitation, temperature, pressure, light, pH, and cell density.

In some embodiments, a culture medium is used to culture a cell. A “culture medium” or “growth medium” as used herein refers to a mixture of components that supports the growth of cells. In some embodiments, the culture medium may exist in a liquid or solid phase. A culture medium of the present disclosure can contain any nutrients required for growth of cells. The growth medium may also contain any compound used to modulate the expression of a nucleic acid, such as one operably linked to an inducible promoter (for example, when using a yeast cell, galactose may be added into the growth medium to activate expression of a recombinant nucleic acid operably linked to a GAL1 or GAL10 promoter). In further embodiments, the culture medium may lack specific nutrients or components to limit the growth of contaminants, select for microorganisms with a particular auxotrophic marker, or induce or repress expression of a nucleic acid responsive to levels of a particular component.

In some embodiments, the methods of the present disclosure may include culturing a host cell under conditions sufficient for vector expression. Suitable culture media and conditions may differ among different cells depending upon the biology of each cell. Suitable culture media and conditions may also differ based upon the conditions under which a given promoter, e.g., an RNA polymerase III promoter, is active. Selection of a culture medium, as well as selection of other parameters required for growth (e.g., temperature, pH, oxygen levels, pressure, light, etc.), suitable for a given cell based on the biology of the cell are well known in the art. Examples of suitable culture media may include, without limitation, common commercially prepared media, such as Yeast Extract Peptone Dextrose broth (YEPD or YPD), Luria Bertani (LB) broth, Sabouraud Dextrose (SD) broth, or Yeast medium (YM) broth. In other embodiments, alternative defined or synthetic culture media may also be used.

Many techniques known in the art allow the detection of vector expression. In some embodiments, vector expression may be determined by direct detection of encoded RNA and/or protein using techniques including, without limitation, nucleic acid/protein purification, Northern blotting, Western blotting, immunoprecipitation, in situ hybridization, RNA sequencing, or PCR amplification followed by electrophoretic mobility assay or nucleotide sequencing (e.g., of a DNA barcode). In some embodiments, vector expression may be determined by inference of expression based upon a discernible phenotype (e.g., growth upon antibiotic treatment when a selectable marker is expressed or growth under normally auxotrophic conditions when an auxotrophic marker is expressed).

As used herein, the terms “polynucleotide,” “nucleic acid,” “oligonucleotide,” and “nucleotide” may be used interchangeably and refer to a sequence of nucleotides linked by phosphodiester bonds. Unless specified, a nucleic acid may generically refer to ribonucleic acid or deoxyribonucleic acid.

As used herein, the terms “polypeptide” and “protein” may be used interchangeably and refer to a sequence of amino acids linked by peptide or amide bonds.

All publications, patents, and patent applications cited herein are hereby incorporated by reference in their entirety for all purposes.

The following example is offered for illustrative purposes and to aid one of skill in better understanding the various embodiments of the disclosure. The following example is not intended to limit the scope of the present disclosure in any way.

### EXAMPLES

Described herein is the engineering of a portable and modular Cas9 genome editing system for yeast. This system contains a uniquely engineered bi-functional synthetic single guide (sgRNA), which resulted in the high efficiency genesis of DNA insertions in industrial polyploid yeast strains.

As part of this system, a plasmid-based screen that allows for a very simple and high throughput genome editing protocol has also been developed. This system has been optimized for use in Saccharomyces cerevisiae and was able to achieve 100% editing efficiency when Cas9+sgRNA and a linear DNA molecule were co-transformed. The efficiency is so high that the requirement of antibiotic resistance markers to identify integrated DNA in the yeast genome was eliminated. This system has been used to edit the genomes of a prototrophic haploid, a diploid and two industrial polyploid yeast strains. This technology can be applied to laboratory, wild and industrial yeasts without any previous genetic modification to the organism. The results described herein demonstrate that this reagent set and protocol are capable of large and small gene deletions, as well as gene insertions, including inserting genes from other organisms into a yeast genome.

The Cas9 technology described herein enables rapid engineering of non-domesticated yeast strains that are important for industrial applications. Further, the ability to make multiplex mutations with high efficiency allows for the genetic analysis of complex traits on a scale that was previously impossible.

### Materials and Methods

Cloning the pCAS Plasmid Backbone

Gibson Assembly Mastermix (E2611L) (New England Biolabs, Ipswich, Mass.) (Gibson, D. G., et al. (2009) Nat. Methods 6(5):343-5) was used to fuse the KANMX (Available online at Yeastdeletionpages.com) cassette to the pUC bacterial origin of replication from pESC-URA (Agilent Technologies, Santa Clara, Calif.). Restriction-free (RF) cloning (van den Ent, F. and Lowe, J. (2006) J. Biochem. Biophys. Methods 67(1):67-74) was used to add a yeast 2μ origin of replication from pESC-URA to the pCAS backbone. The resulting pCAS backbone plasmid was propagated in yeast to confirm functionality.

### Cas9 Expression Constructs

The Cas9 gene from Streptococcus pyogenes was amplified from clone MJ824 (Jinek, M., et al. (2012) Science 337(6096):816-21) and cloned into the pCAS backbone plasmid by RF cloning. A yeast nuclear localization signal (NLS) sequence, codon optimized using IDT software (Integrated DNA Technologies, Coralville, Iowa), was then cloned into the plasmid by RF cloning. Additional elements fused by RF cloning to the Cas9-NLS sequence included the GFP gene, the CYC1 terminator from S. cerevisiae strain S288c (Available online at Yeastgenome.org) and the promoters from the genes TDH3, TEF1, RNR2 and REV1, also taken from strain S288C (Lee, M. E., et al. (2013) Nucleic Acids Res. 41(22):10668-78). For genome editing experiments, the GFP sequence was removed from the Cas9 gene and replaced with a C-terminal His₈ affinity tag, by RF cloning.

### Cas9-GFP Localization and Expression

Expression and localization of Cas9-GFP was verified by imaging haploid prototrophic S. cerevisiae S288c cells transformed with pCas9-GFP::KAN using fluorescence microscopy (Leica Epifluorescence, Leica Microsystems, Buffalo Grove, Ill.). Cells were grown overnight and nuclear localization visualized at 100× magnification.

Engineering of sgRNA Constructs

Synthetic DNA (Integrated DNA Technologies, Coralville, Iowa) for the sgRNA and for a catalytically active form of the Hepatitis Delta Virus (HDV) Ribozyme was sequentially cloned by RF cloning into the Cas9 containing vector. The terminator (200 bp) of SNR52 (Available online at Yeastgenome.org) was cloned 3′ of the ribozyme-sgRNA sequence by RF cloning. Pol III promoters were PCR amplified from S288c genomic DNA and cloned 5′ of the ribozyme-sgRNA sequence by RF cloning. The tRNA promoters included the full-length tRNA plus 100 base pairs upstream of the tRNA gene. The sgRNAs used for multiplex targeting were PCR amplified using primers containing 5′ and 3′ restriction sequences and sub-cloned into pCAS by ligation dependent cloning into SalI, SpeI and SacII unique restriction sites.

### Fitness Analysis of Cas9 Expressed by Different Promoters

Yeast cells containing pCAS (Cas9-His₈ variant) were grown in a Bioscreen C Growth Curve Analyzer (Growth Curve USA, Piscataway, N.J.) in 200 μL of YPD+G418 (200 mg/L) liquid medium (20 g/L Peptone (Bacto 211667), 10 g/L Yeast Extract (Bacto 212750), 0.15 g/L Adenine hemisulfate (Sigma A9126) and 20 g/L Glucose (Sigma G8270)+G418 (Santa Cruz Biotechnology 29065A). Cells were grown in five biological replicates each with five technical replicates for 48 hours at 30° C. under constant shaking. The wild-type control containing an empty vector was also grown in five technical replicates. Mean and standard deviations of the optical density at 600 nm were calculated for each time point measured by the Bioscreen.

qRT-PCR of sgRNAs

Cells containing the pCAS plasmid with sgRNA inserts were grown in 900 μL of YPD+G418 medium for 24 hours at 30° C. and 750 rpm. Total RNA was extracted from exponentially growing yeast cells using Ambion RNA RiboPure™ Yeast Kit (AM1926)(Life Technologies, Carlsbad, Calif.). RT-qPCR was performed on the Applied Biosciences StepOne™ Real-Time PCR System (Applied Biosystems, Foster City, Calif.) using the Invitrogen EXPRESS One-Step SYBR® GreenER™ Kit (Life Technologies, Carlsbad, Calif.). The RT-qPCR expression level data was quantified using the Comparative CT_{T} (ΔΔC_{T}) method and relative abundance of the sgRNA was normalized to the mRNA transcript UBC6, which was used as the endogenous control. The primers sequence used for the RT reaction was 5′-AAAAGCACCGACTCGGT-3′ and the additional q-PCR primer used was 5′-GTTTTAGAGCTAGAAATAGCAAG-3′. The primers used for the UBC6 endogenous control were (RT) 5′-CATTTCATAAAAAGGCCAACC-3′ and qPCR 5′-CCTAATGATAGTTCTTCAATGG-3′.

### CRISPR-Cas9 Screening Protocol

The Cas9 transformation mix included 90 μL yeast competent cell mix (OD₆₀₀=1.0), 10.0 μL ssDNA (Sigma D9156, St. Louis, Mo.), 1.0 μg pCAS plasmid, 5.0 μg of linear repair DNA and 900 μL Polyethyleneglycol₂₀₀₀ (Sigma), 0.1 M Lithium acetate (Sigma) 0.05M Tris-HCl and EDTA. To measure Cas9 independent integration, the linear DNA was co-transformed with a plasmid lacking the Cas9 protein and sgRNA (pOR1.1). Cells were incubated 30 minutes at 30° C., and then subjected to heat shock at 42° C. for 17 minutes. Following heat shock, cells were re-suspended in 250 μL YPD at 30° C. for two hours and then the entire contents were plated onto YPD+G418 plates (20 g/L Peptone, 10 g/L Yeast Extract, 20 g/L Agar, 0.15 g/L Adenine hemisulfate, 20 g/L Glucose and G418 at 200 mg/L). Cells were grown for 48 hours at 37° C., imaged using the Biorad ChemiDoc Imager (Biorad, Hercules, Calif.) and replica plated onto phenotype-selective media.

URA3 mutants were selected on 2.0 g/L Yeast nitrogen base without amino acids or ammonium sulfate (Sigma Y1251), 5.0 g/L Ammonium sulfate (Sigma A4418), 1.0 g/L CSM (MP Biosciences 4500-012), 20 g/L Glucose, 20 g/L Agar+5-fluoroorotic acid (lg/L) (Goldbio F-230-25); LYP1 mutants were selected on 2.0 g/L Yeast nitrogen base without amino acids or ammonium sulfate, 5.0 g/L Ammonium sulfate, 1.0 g/L CSM-lysine (MP Biosciences 4510-612), 20 g/L Glucose, 20 g/L Agar+thialysine (100 mg/L) (Sigma A2636); CAN1 mutants were selected on 2.0 g/L Yeast nitrogen base without amino acids or ammonium sulfate, 5.0 g/L Ammonium sulfate, 1.0 g/L CSM-arginine (MP Biosciences 4510-112), 20 g/L Glucose, 20 g/L Agar+canavanine sulfate (50 mg/L) (Sigma C9758); the remaining auxotrophic mutants were selected on 2.0 g/L Yeast nitrogen base without amino acids or ammonium sulfate, 5.0 g/L Ammonium sulfate, 1.0 g/L CSM, 20 g/L Glucose, 20 g/L Agar; and aerobic respiration deficient mutants (petites) were selected on 20 g/L Peptone, 10 g/L Yeast Extract, 20 g/L Agar, 0.15 g/L Adenine hemisulfate, 20 g/L Glycerol (Sigma G5516).

Colonies from the YPD+G418 plates were picked and grown overnight in 1 mL of YPD. Genomic DNA was extracted from these cultures using the MasterPure Yeast DNA Extraction Kit (Epicentre MPY80200). PCR confirmation of the 60-mer integration allele was performed using primers flanking the target site. PCR products were purified by Exo-SAP-IT (Affymetrix 78201) and Sanger sequenced to confirm barcode sequence in the amplicon.

### Multiplex Genome Targeting by Cas9

Multiplex targeting was performed as described using pCAS plasmids containing more than one sgRNA expression construct cloned into one of the restriction sites by ligation dependent cloning. Single versus double mutant efficiency was scored relative to the number of colonies present on the YPD+G418 plate. Genomic DNA isolation and PCR of the integration site was performed as described.

### Multiplex In Vivo Assembly of DNA Using Cas9

Drug resistance cassettes were assembled in vivo from three linear double-stranded DNA fragments PCR amplified from the Ashbya gosipii TEF1 promoter (AgP_{TEF1}), the nourseothricin open reading frame (Nat^{R}) and Ashbya gosipii TEF1 (AgT_{TEF1}) terminator in separate reactions. The primers used to amplify the promoter and terminators contained 50 bp of homology to the nourseothricin ORF and 50 bp of homology to the genomic target.

The cellobiose utilization pathway was assembled in vivo by using two sets of three PCR-amplified linear dsDNA fragments individually including the SCP_{PGK1} promoter, the N. crassa cdt-1 open reading frame and ScT_{CYC1} terminator (for the cdt-1 gene), or the ScP_{TDH3} promoter, the N. crassa gh1-1 open reading frame and ScT_{ADH1} terminator (for the gh1-1 gene). The primers used to amplify the promoters and terminators contained 50 bp of homology to either the cdt-1 or gh1-1 ORFs and 50 bp of homology to the respective the genomic targets.

Five micrograms of each DNA molecule were co-transformed with the pCAS plasmid and screened for G418 resistance as described above. Colonies containing the desired phenotypes following replica plating: either (a) drug resistance (nourseothricin 100 mg/L) (Goldbio N-500-1); or (b) cellobiose utilization (5% cellobiose) (Fluka 22150) were compared to the number of colonies on the YPD+G418 to determine efficiency of multiplex assembly.

### Error-Prone PCR of the Cellodextrin Transporter CDT1

To generate CDT1 mutant allele libraries, the GeneMorph II Random Mutagenesis Kit (Aglient 200550)(Agilent Technologies, Santa Clara, Calif.) was used to amplify the N. crassa cdt-1 open reading frame. The library of cdt-1 mutant alleles was co-transformed with the SCP_{PGK1} promoter and ScT_{CYC1} terminator into a yeast strain containing a previously-intergrated gh1-1 gene. Approximately 2000 colonies were pooled and resuspended in minimal cellobiose medium (SC) (2.0 g/L Yeast nitrogen base without amino acids or ammonium sulfate, 5.0 g/L Ammonium sulfate, 1.0 g/L CSM, 20 g/L Cellobiose). Resuspended cells were immediately spread evenly on SC plates, which were the TO samples. Ten microliters of cells were inoculated in 50 mL of SC medium in biological triplicate. Cells were harvested after five days and spread onto SC plates. Cells were grown at 30° C. for four days. In total, 132 colonies were selected from the SC plates and arrayed in a 96 well format for further analysis.

### Tecan Growth Analyzer and Fitness Calculation of Cdt1^{S209}

Cells were grown overnight in 1 mL of Synthetic Dextrose (2%) (SD) in 96 well plates. Cultures were diluted 1:500 in SC (4%) and 150 μL were grown using the Tecan Sunrise (Tecan Systems Inc., San Jose, Calif.) in biological triplicate for four days at 30° C. Average and standard deviation was calculated for each biological sample. Relative fitness was calculated by measuring area between the curve (ABC) for cdt1^{S209} and cdt1 containing cells relative to wild type (cdt-1⁻) cells (ABC=AUC cdt1⁺-AUC cdt-1⁻). Percent cellobiose utilization capacity is equal to (AUC cdt1^{S209}/AUC cdt1)*100.

### CDT Transporter Activity Assay

CDT transporter assay was performed as described in Galazka, J. M., et al. (2010) Science 330(6000):84-6.

### Illumina HiSeq Sequencing of Off-Target Mutations and Sequence Alignments

Whole genome sequencing was performed by the UC Davis Genome Center (Davis, Calif.) using the Illumina MiSeq platform (Illumina, Hayward, Calif.) to produce 150 bp paired-end reads. The software package versions used for sequencing data analysis were as follows: BWA (v. 0.7.5a-r405), Picard (v. 1.92(1464)), SAMtools (v. 0.1.19-44428cd) and the GATK (2.7-2-g6bda569). The S288C reference genome (v. R64-1-1, release date Feb. 3, 2011) was obtained from the Saccharomyces Genome Database (yeastgenome.org) and prepared for use in sequencing data analysis with bwa index, CreateSequenceDictionary from Picard, and samtools faidx. Sequencing reads were processed with Scythe (v. 0.991) to remove adapter contamination and Sickle (v. 1.210) to trim low quality bases. Processed reads were mapped to the S288C reference genome using bwa mem with the -M option for picard and GATK compatibility. The mapped reads were sorted with SortSam and duplicate reads were marked with MarkDuplicates from Picard. Read alignments were refined by performing local realignment with the RealignerTargetCreator and IndelRealigner walkers from the GATK on all samples collectively. Variant detection for both SNPs and INDELs was performed with GATK's UnifiedGenotyper, with parameters adjusted for haploid genomes and no downsampling of coverage, for each sample independently. The resulting SNP and INDEL calls were filtered with the VariantFiltration walker from GATK (see header of the VCF file, supplemental VCF file, for details).

A custom perl script was written to identify all GG dinucleotide sequences in the S288C reference genome, extract every Cas9 target sequence (i.e. 23nt sequence corresponding to the “NGG” PAM site plus 20 nucleotides immediately 5′ of the PAM site), and obtain the genome coordinates ranging from four nucleotides 5′ of the PAM site to the end of the PAM site (i.e. the Int upstream of the end of the PAM site), which encompasses the region where Cas9 creates a double strand cut (supplemental BED file). Cas9 target sequences were added to VCF files as custom annotations using snpEff (v3.3h), and SnpSift (v3.3h) was used to extract desired fields into tables for analysis with custom R scripts. Needleman—Wunsch global alignments between guide sequences and Cas9 target sequences were performed using the pairwiseAlignment function (Biostrings package, Bioconductor) in R, with a substitution matrix of −1 for mismatches and 2 for matches, produced with the nucleotideSubstitutionMatrix function (Biostrings package, Bioconductor). The probability of there being a better match for the guide sequence to a given Cas9 target sequence was calculated as the frequency of Cas9 target sequences with better alignments to the same guide sequence, amongst 10,000 randomly selected Cas9 target sequences. To compile counts of all variants and various subclasses, a GATKReport was generated from the VCF files with GATK's VariantEval walker, read into R using the “gsalib” library, and the desired categories were extracted with a custom R script.

URA3 and LYP1 targeted strains were sequenced and searched for newly risen SNPs and INDELs within 4nt upstream of any PAM site in the genome, to the end of the same PAM site. Eleven distinct variant sites were identified across the nine URA3- and LYP1-targeted strains (Fig d2.tab, d4bMaxs.tab or d5.tab or d6.tab).

For the 25 Cas9 target sequences whose PAM site was within 4nt downstream of a detected variant, there were at most 10 out of 23 nucleotide matches in end-to-end alignments with our guide sequences. Given this lack of alignment, it is thought to be highly unlikely that the URA3- and LYP1-guide sequences directed Cas9 to any of these target sequences. To evaluate the likelihood that URA3 or LYP1 guide sequences actually did target Cas9 to any of the sites where these 11 variants were found, local alignments of guide sequences were performed to all Cas9 target sequences whose PAM site was within 4nt downstream of a detected variant, as well as to 10,000 randomly selected Cas9 target sequences from the genome. Since guide sequences are expected to have a better match to 13% or more of all Cas9 target sequences (˜126,000 or more sites) than to the best matching Cas9 target sequence with a nearby variant (d4bMaxs.tab or d5.tab or d6.tab), and the number of nucleotide matches in end-to-end alignments is at most 10, the variants identified in the genomes of URA3- and LYP1-targeted strains are considered highly unlikely to be the result of off-target Cas9 modifications.

### Results

### Example 1

### Engineering a Dual Function sgRNA and a Cas9 Protein for Yeast

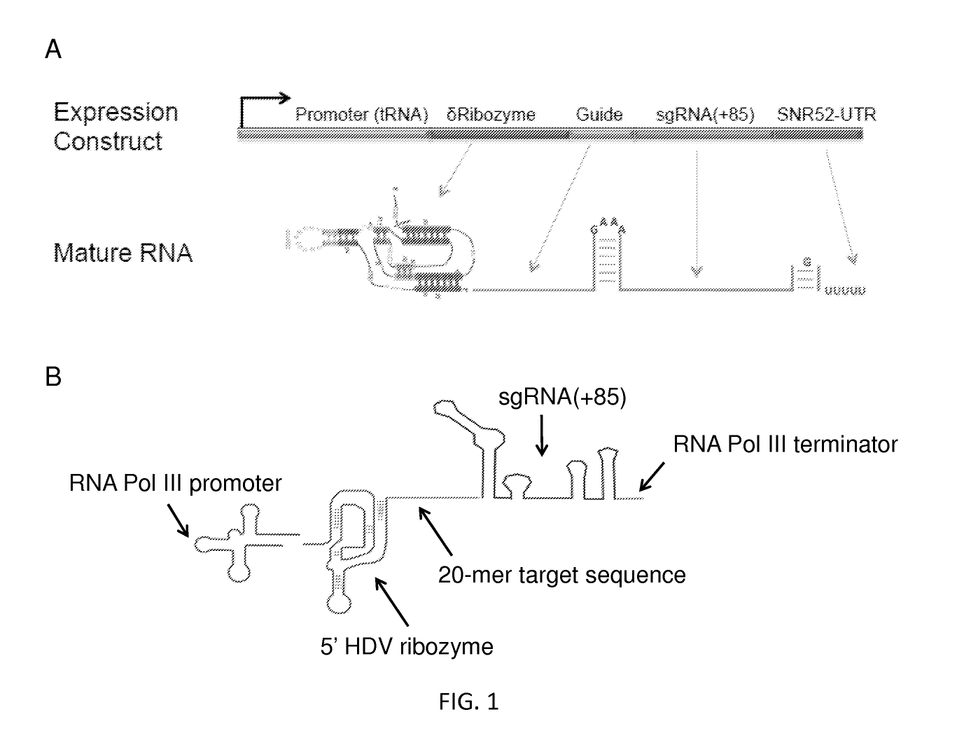

This Example describes a dual function sgRNA and Cas9 protein. FIG. 1A provides a diagram of an exemplary dual function sgRNA. The sgRNA(+85) variant was used for the sgRNA component (Mali, P., et al. (2013) Science 339(6121):823-6). A catalytically active self-cleaving delta ribozyme from the Hepatitis D virus was fused 5′ to the guide and sgRNA(+85) sequences using a UU dinucleotide linker. The ribozyme enzymatically cleaves the RNA immediately 5′ to its coding sequence, thereby removing any 5′ RNA that precedes the ribozyme (Ke, A., et al. (2007) Structure 15(3):281-7; Webb, C. H., et al. (2009) Science 326(5955):953). This allowed for the use of tRNAs as promoters for RNA polymerase III to express the sgRNA used for Cas9 targeting, because the tRNA will be removed. The tRNA may be removed because the RNA Polymerase III binding motifs are found within the tRNA itself (Orioli, A., et al. (2012) Gene 493(2):185-94).

Following transcription, the ribozyme folds into its catalytically active form and auto-catalyzes the removal of the tRNA promoter (FIG. 1B). The sgRNA and tRNA dissociate from each other so the sgRNA remains in the nucleus to bind with the Cas9 protein, and the tRNA is exported for protein biosynthesis. Because there are a relatively limited number of RNA Polymerase III promoters that are not tRNAs, the ribozyme-sgRNA fusion RNA is thought to enable the use of all of the RNA Polymerase III promoters, including all of the tRNA variants, for expression of the sgRNA. This greatly increases the number of promoters available for expressing the sgRNA and is thought to allow for a broader and more sensitive spectrum of expression levels for the sgRNA.

Moreover, the transcriptional terminator for RNA Pol III promoters is simple and thought to be universal amongst eukaryotes. A short string of uridine nucleotides, typically 5 or 6 in Ascomycetes, is sufficient for transcriptional termination (Marck, C., et al. (2006) Nucleic Acids Res 34(6):1816-35). The termination sequence 5′-UUUUUUUTUUUUUU-3′ was used for the sgRNA construct.

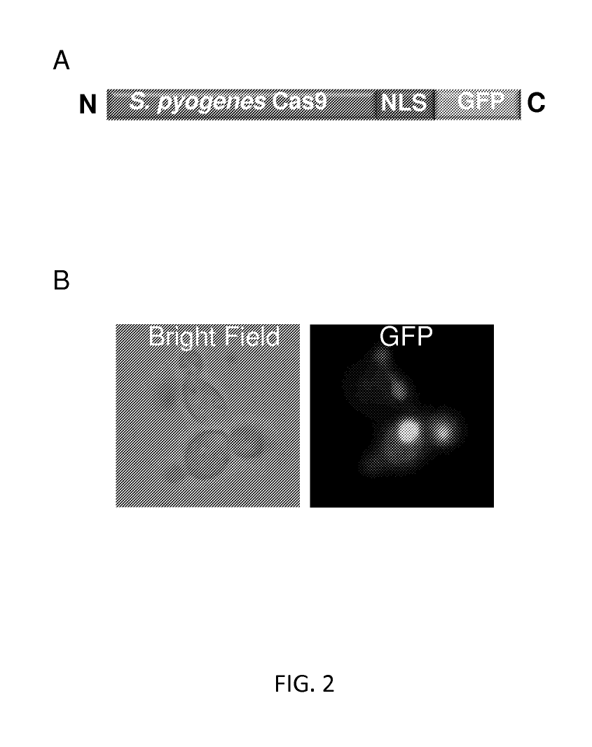

A Cas9 protein was also engineered to allow use in yeast cells. FIG. 2A illustrates this Cas9 construct. Briefly, the Cas9 gene was amplified from Streptococcus pyogenes. A polynucleotide encoding a yeast nuclear localization sequence (NLS) was codon optimized and fused 3′ to the Cas9 coding sequence. A GFP coding sequence was fused 3′ to the NLS sequence. To regulate expression of Cas9 in yeast, the construct shown in FIG. 2A was further linked to the CYC1 terminator from S. cerevisiae strain S288c and one of a variety of promoters, including TDH3, TEF1, RNR2 and REV1. This construct was transformed into haploid S288C S. cerevisiae cells. As shown in FIG. 2B, the engineered Cas9 protein localized to the nucleus in yeast cells.

These results demonstrate the creation of an sgRNA-Cas9 system suitable for use in yeast cells.

### Example 2

### The Presence of a Ribozyme Increases the Relative Cellular Abundance of sgRNA

This Example demonstrates that the presence of a ribozyme is able to increase the relative cellular abundance of sgRNA.

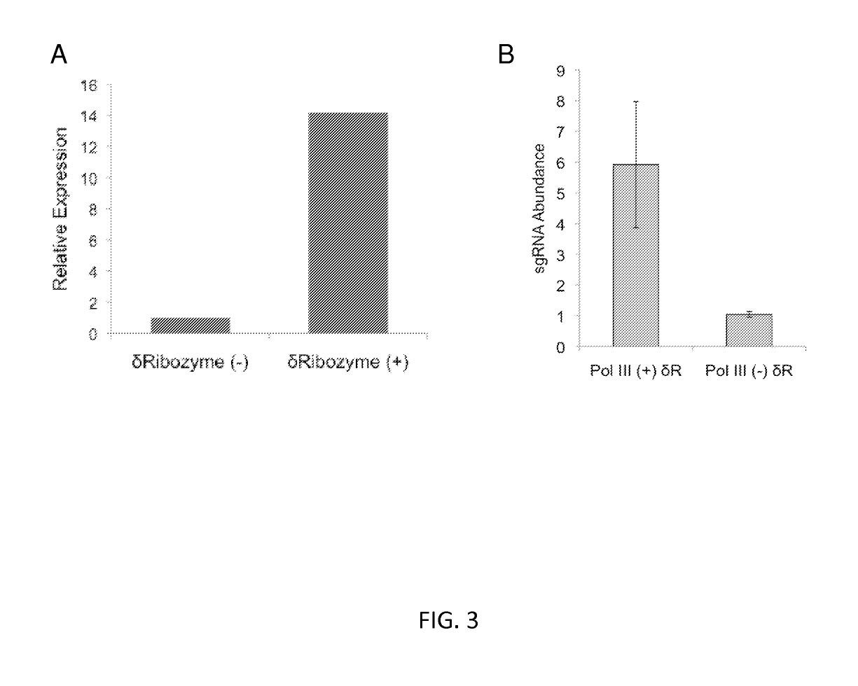

Using the promoter for TDH3 (an RNA Polymerase II promoter), sgRNA was expressed with and without the 5′ ribozyme, and the abundance of sgRNA was measured using quantitative real-time PCR (qRT-PCR). As shown in FIG. 3A, the relative abundance of sgRNA was increased approximately 15-fold when the 5′ ribozyme was fused.

To confirm these results are applicable to RNA Pol III promoters, the tyrosine tRNA promoter was also used to drive sgRNA expression, with and without the 5′ ribozyme. As shown in FIG. 3B, the relative abundance of sgRNA was increased approximately 6-fold when the 5′ ribozyme was fused, demonstrating that the 5′ ribozyme system is also useful for RNA Pol III promoters.

This Example demonstrates that a 5′ ribozyme fused to sgRNA increases the cellular abundance of the sgRNA in both RNA Pol II and RNA Pol III promoters. Without wishing to be bound to theory, it is thought that the abundance of cellular sgRNA may often be rate limiting for Cas-mediated genome editing, so the dual function ribozyme-sgRNA described herein may facilitate more complex and/or multiplex reactions in which sgRNA may become the rate-limiting component.

### Example 3

### A Cas9-Dual Function sgRNA System for Targeted Genome Editing

This Example describes how the dual function sgRNA may be used for targeted genome editing in yeast.

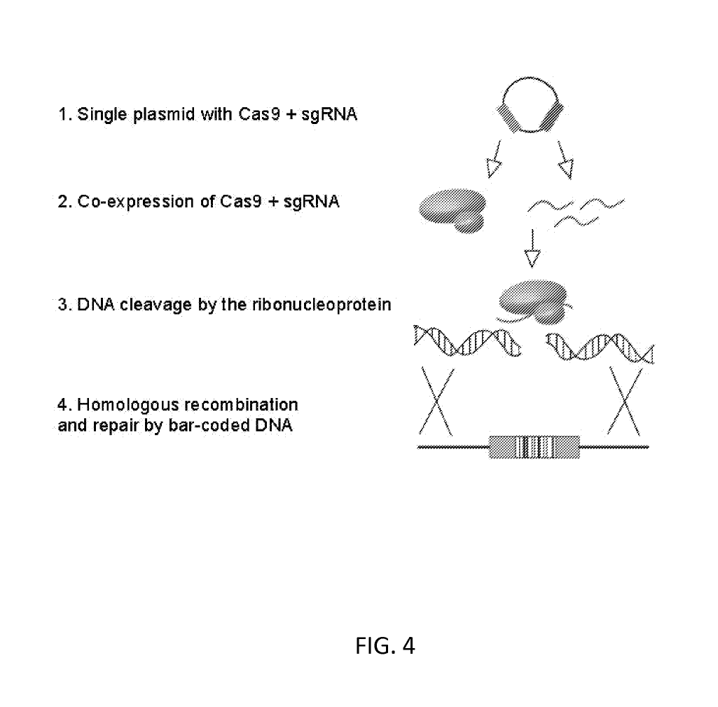

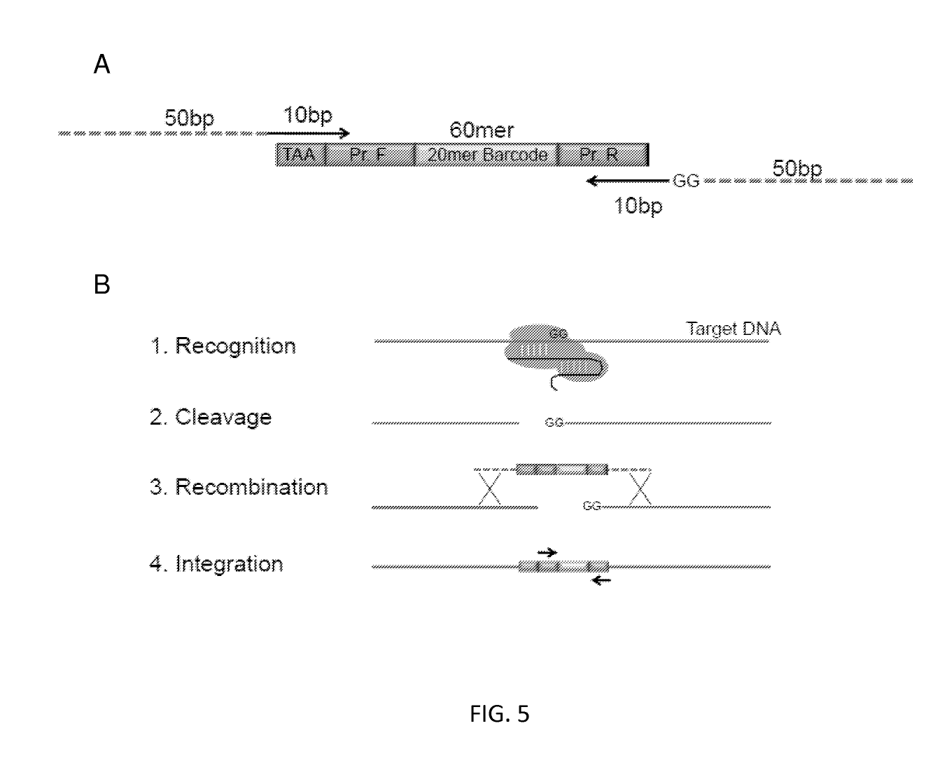

FIG. 4 provides an exemplary overview of a Cas9-dual function sgRNA system for genome editing. Cas9 protein and sgRNA are co-expressed from a single plasmid with a linear barcode oligonucleotide (FIG. 5A). The linear oligonucleotide acts as a template for DNA repair, resulting in an insertion allele. The barcode DNA contains a STOP codon, two common primer sites and a unique 20 nucleotide barcode. The barcode DNA was PCR amplified to add 50 base pairs of homology corresponding to the DNA sequence flanking the genome target site. These 50 bp were used to facilitate homologous recombination of the barcode DNA into the chromosome. For loss-of-function genetic studies the barcode DNA has been integrated, but much larger, linear DNA molecules, e.g., genes that confer drug resistance phenotypes, have also been inserted into the genome.

FIG. 5B provides an exemplary overview of genome editing by integration of the linear barcode oligonucleotide. Cas9 binds to the sgRNA containing a specific 20-mer target sequence. This target sequence is used by the Cas9-sgRNA ribonucleoprotein to recognize genomic DNA sequence identical to the target sequence in the sgRNA. Cas9 then creates a double-stranded break in the chromosome. Repair DNA, e.g., the linear barcode DNA, recombines into the genome using the 50 base pairs of homologous sequence proximal to the cleavage site. A loss of function allele is created where the barcode DNA integrates into the genome.

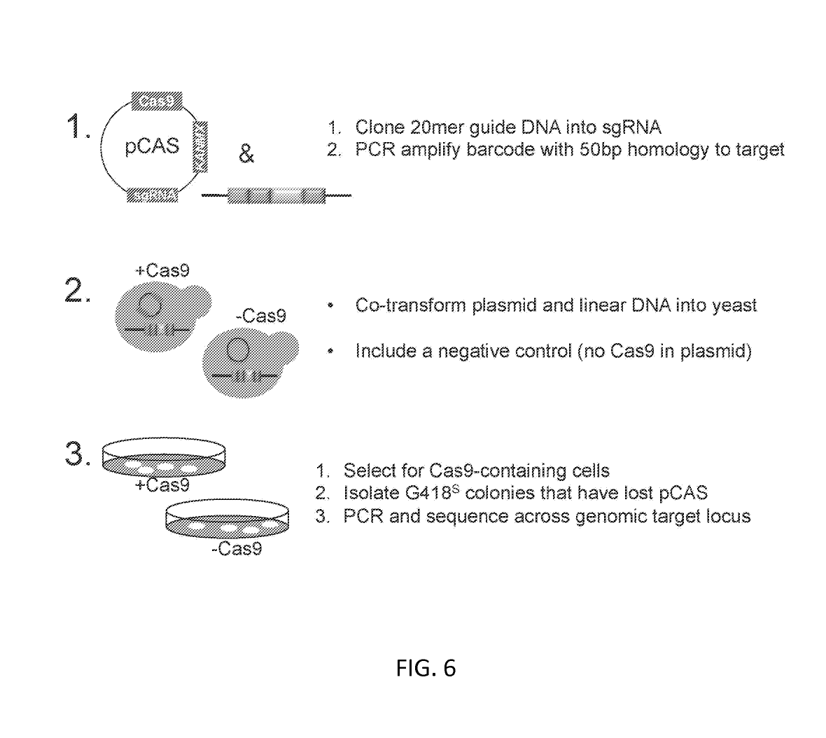

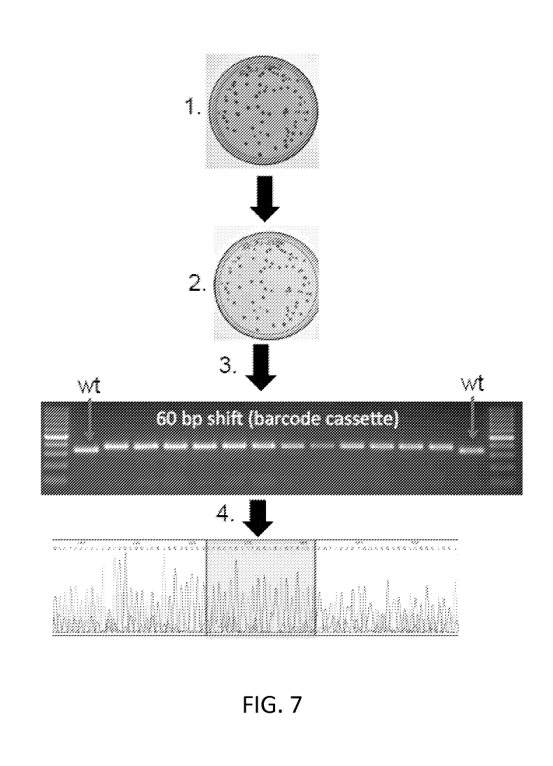

A yeast screening method was developed to test this genome editing system. FIG. 6 provides an exemplary overview of the yeast screening method, and the results are shown in FIG. 7. Briefly, a plasmid containing Cas9, an sgRNA, and the KANMX selection marker was co-transformed into yeast along with a linear barcode DNA. Potential transformants were plated onto YPD containing G418 to select for the presence of the plasmid and allowed to grow for 48 hours at 37° C. (step 1 in FIG. 7). Cells were then replica plated onto a selective medium to determine the efficiency of targeting a specific locus. In this example, the URA3 locus was targeted, and cells were replica plated onto medium containing 5-Fluoroorotic acid (5-FOA) (step 2 in FIG. 7). As a negative control, the same transformations were also carried out using a plasmid lacking Cas9. Barcode DNA was then PCR amplified from genomic DNA to determine whether the transformed cells contained the barcode at the appropriate locus (step 3 in FIG. 7). To confirm the presence of the 20-mer barcode, this PCR product was sequenced (step 4 in FIG. 7).

### Example 4

### Using the Yeast Screening Model to Test Parameters for a Cas9-Dual Function sgRNA System

This Example describes how the yeast screening model described in the previous Example was used to test the function of a Cas9-dual function sgRNA system for yeast genome editing.

First, the effect of the ribozyme was tested by comparing the targeting efficiency of an sgRNA containing a 5′ ribozyme to the targeting efficiency of an sgRNA lacking a 5′ ribozyme, using haploid yeast cells. SNR52 was used as a promoter for the dual function sgRNA. Targeting efficiency was tested at two distinct genetic loci: URA3 and LYP1. These loci provide a clear phenotype for assessing the successful creation of loss of function mutations due to selection phenotypes when cells are grown on 5-FOA or thialysine, respectively. After co-transformation, genomic DNA was extracted, and PCR amplification was performed across the target loci using unique primer sequences adjacent to the target site in the genome. In this experiment, a 60 base pair shift in DNA mobility indicates a successful integration. PCR products were then sequenced to identify the barcode sequence.

The efficiency of targeting was found to be 100%, regardless of the presence of the 5′ ribozyme. These results suggest that in haploid yeast, using the SNR52 promoter to drive expression, integration is optimally efficient, regardless of the presence of a 5′ ribozyme.

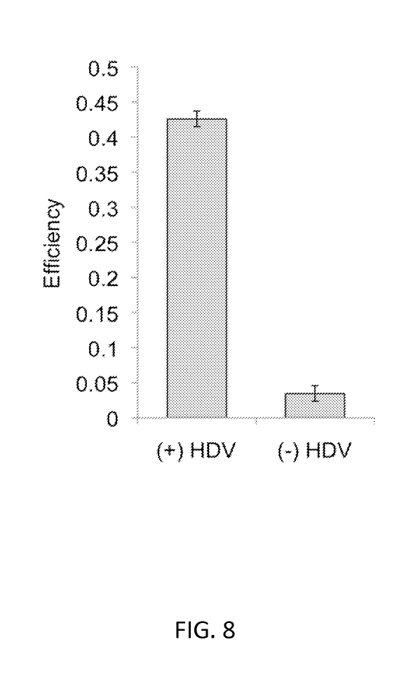

However, very different results were obtained when targeting two genomic loci simultaneously in diploid S. cerevisiae (S288C) cells. A single plasmid containing one copy of Cas9 and two sgRNAs (specific for URA3 and LYP1) was used to target both loci simultaneously. As shown in FIG. 8, in this system, the presence of a 5′ ribozyme (hepatitis delta virus ribozyme, a.k.a. HDV or ∂R) resulted in a 12-fold increase in duplex targeting efficiency, as compared to using an sgRNA lacking a 5′ ribozyme (targeting efficiency was 43% with the 5′ ribozyme and 3.5% without the 5′ ribozyme). Without wishing to be bound to theory, a more abundant pool of sgRNA may be advantageous for more complex or multiplex genome editing experiments, and the 5′ ribozyme may boost editing efficiency by increasing the abundance of this pool.

In Ascomycete fungi, RNA Polymerase III (RNA Pol III) controls the expression of all tRNAs, the U6 snRNA (SNR6), RNase P (RPR1), the RNA component of the Signal Recognition Particle (SCR1) and a single snoRNA (SNR52) (Orioli, A., et al. (2012) Gene 493(2):185-94). Ribozyme-sgRNA constructs using each of the four RNA PolIll promoters (SNR52, SNR6, SCR1 and RPR1) and a number of tRNA promoters were next examined. For these experiments, Cas-mediated targeting of the URA3 locus was performed in diploid yeast cells.

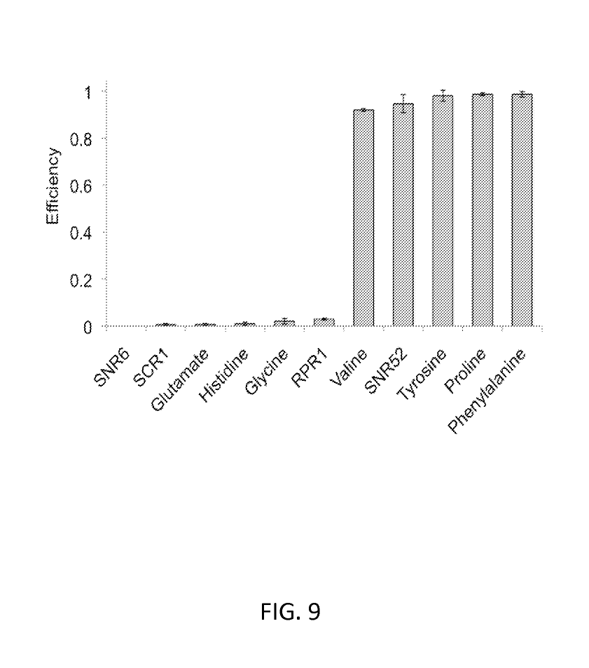

As shown in FIG. 9, one non-tRNA promoter was able to efficiently generate homozygous mutants: SNR52. Several tRNAs, however, were efficient at targeting URA3, including the tRNAs for valine, tyrosine, proline and phenylalanine. These results demonstrate that multiple RNA Pol III promoters, including several tRNAs and a snoRNA, allow efficient genome editing in diploid yeast cells.

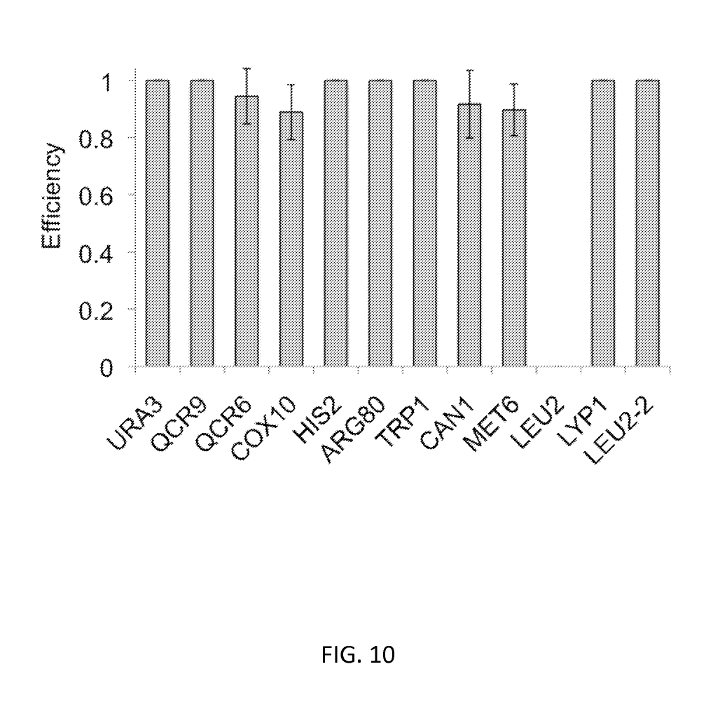

The targeting efficiency for this system at multiple genetic loci was also tested. Different sgRNAs were used to target different, selectable loci in yeast. As shown in FIG. 10, this system showed 100% targeting efficiency at multiple genetic loci (11/13 of the loci tested). In addition, the locus for which targeting did not work (LEU2) was corrected to 100% efficiency by changing the LEU2 guide RNA sequence. These results demonstrate that different sgRNAs allow highly efficient genome editing at multiple loci.

One potential drawback to any genome editing system is the introduction of off-target mutations (i.e., unintended mutations introduced any genetic locus not targeted for editing). In order to evaluate whether the system described above introduces off-target mutations, whole genome sequencing experiments were conducted. Genomes of 5 biological replicates of S. cerevisiae strains in which URA3 was targeted and 4 biological replicates of S. cerevisiae strains in which LYP1 was targeted were sequenced and compared to a wild-type reference strain. Newly risen SNPs and INDELs 30 base pairs of any protospacer adjacent motif (PAM) site functional with S. pyogenes Cas9 (e.g., an NGG dinucleotide) were studied. In total, approximately 108,000,000 bases of sequence were collected.

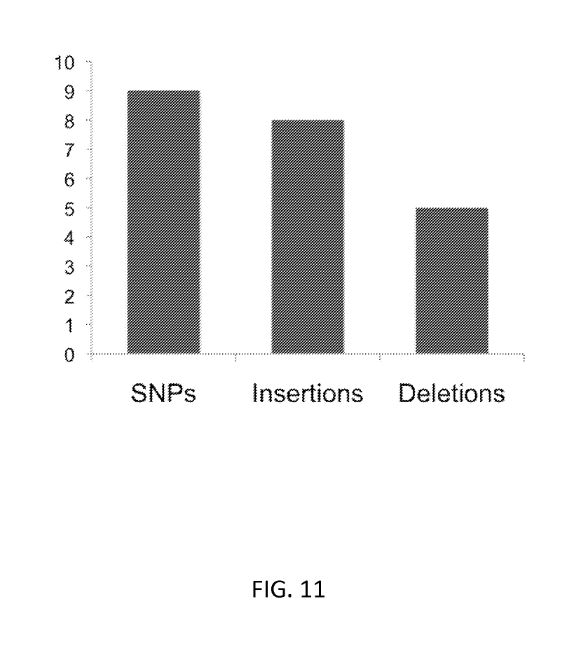

FIG. 11 illustrates the results from this whole genome sequencing study. In total, only a handful of mutations were found adjacent to PAM sites in the entire 12 megabase S. cerevisiae genome, representing eleven distinct variant sites identified across the nine URA3- and LYP1-targeted strains. Local alignments were performed between the guide sequences used and any Cas9 target sequences whose PAM site was within 4nt downstream of a detected variant. The highest alignment identified between a guide RNA and a variant sequence was only 10 out of 23 nucleotide matches (overall, not in succession). Without wishing to be bound to theory, it is thought that 12 or more perfect matches between an sgRNA and the DNA target sequence facilitate Cas9-mediated editing. Given these results, it appears highly unlikely that any of these mutations were targeted by Cas-mediating editing. These results demonstrate that this genome editing system is not likely to cause significant off-target mutagenesis.

### Example 5

### Cas-Mediated Genome Editing in Industrial Polyploid Yeast Cells and Multiplex Genome Editing

This Example demonstrates that the CRISPR-Cas9 genome editing system described in the previous Examples is able to perform efficient genome editing in polyploid and industrial yeast cells. It further demonstrates that this system is able to perform efficient multiplex genome editing.

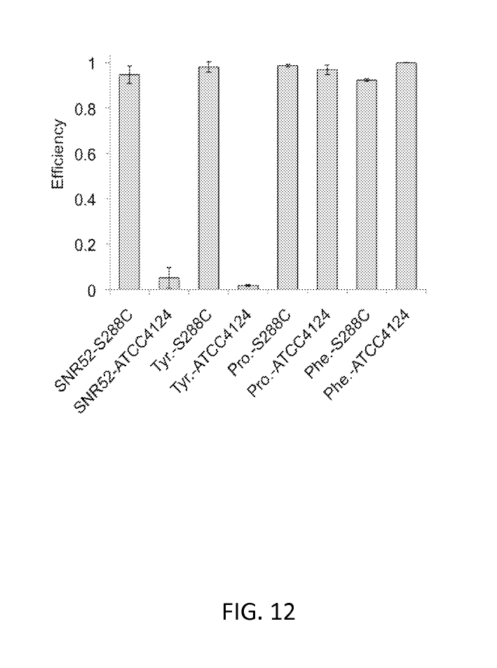

In order to test this system in polyploid yeast used for industrial processes, the strain ATCC4124, which was isolated from a molasses distillery, was used. Targeting efficiency was measured, comparing expression of ribozyme-sgRNA constructs using different RNA Pol III promoters. As shown in FIG. 12, the efficiency of generating a homozygous URA3 mutant was 100% and 97% using the tRNA^{Phe} and tRNA^{Pro} as promoters, respectively. However, the efficiency was only 5% when using the non-tRNA P_{SNR52}. These results suggest that tRNA promoters are able to promote efficient creation of homozygous null mutants in polyploid industrial yeast isolates.

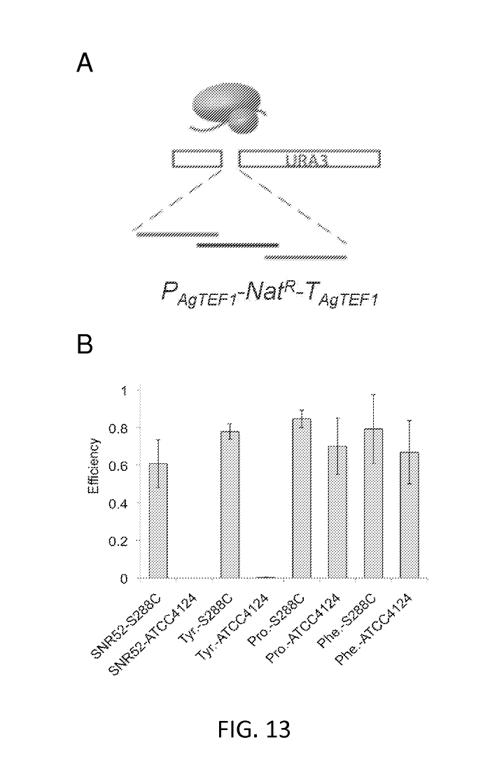

To demonstrate that CRISPR-Cas9 could be used as a cloning platform, the assembly of a functional nourseothricin-resistance (Nat^{R}) gene from multiple PCR products was tested in vivo (FIG. 13A). The correct assembly and insertion of PCR products that encode a transcriptional promoter, protein-coding region and transcriptional terminator result in the expression of the Nat^{R} gene that confers nourseothricin resistance (Krugel, H., et al. (1988) Gene 62(2):209-17). As shown in FIG. 13A, three separate, linear DNA molecules that overlap by 50 base pairs (including the TEF1 promoter and terminator of Ashbya gosypii, and a Nat^{R} drug resistance gene from Streptomyces noursei) were co-transformed, and these polynucleotides were targeted to the URA3 locus using an sgRNA.

FIG. 13B illustrates the efficiency of Cas-mediated integration and assembly of all three DNA fragments to the correct locus, as measured by a combination of 5-FOA^{R} and Nat^{R}. For example, targeting efficiency was 85% in diploid S288C cells and 70% in ATCC4124 cells using the tRNA^{Phe} as the sgRNA promoter. These results demonstrate a novel, one-step, marker- and selection-free method of assembling functional genes in the S. cerevisiae genome, including the genome of an industrial yeast isolate.

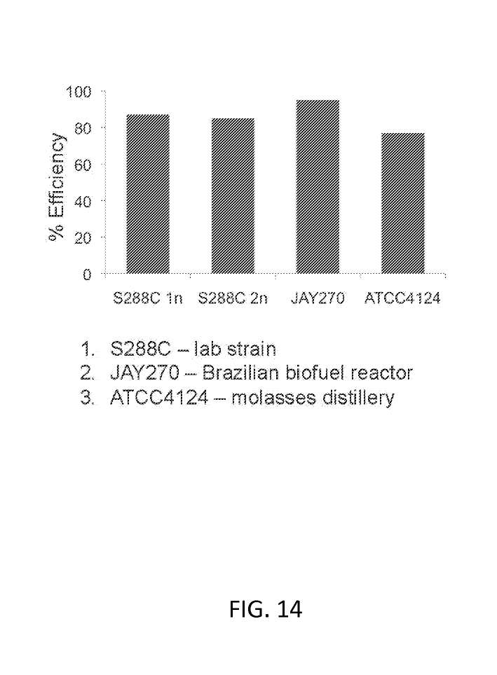

FIG. 14 illustrates an experiment demonstrating that the CRISPR-Cas9 system described herein may be used efficiently as a cloning platform in haploid, diploid, and industrial yeast strains. The TEF1 promoter and terminator of Ashbya gosypii and Nat^{R} drug resistance gene from Streptomyces noursei were used as described above to target the URA3 locus in four yeast strains: haploid S288C, diploid S288C, JAY270 (industrial strain isolated from a Brazilian biofuel reactor), and ATCC4124. As shown in FIG. 14, each of these strains was targeted (i.e., resulted in homozygous replacement of the URA3 locus) at an efficiency between approximately 80-90%. These results demonstrate the utility of the CRISPR-Ca9 system for genome editing in several different and industrially useful yeast strains.

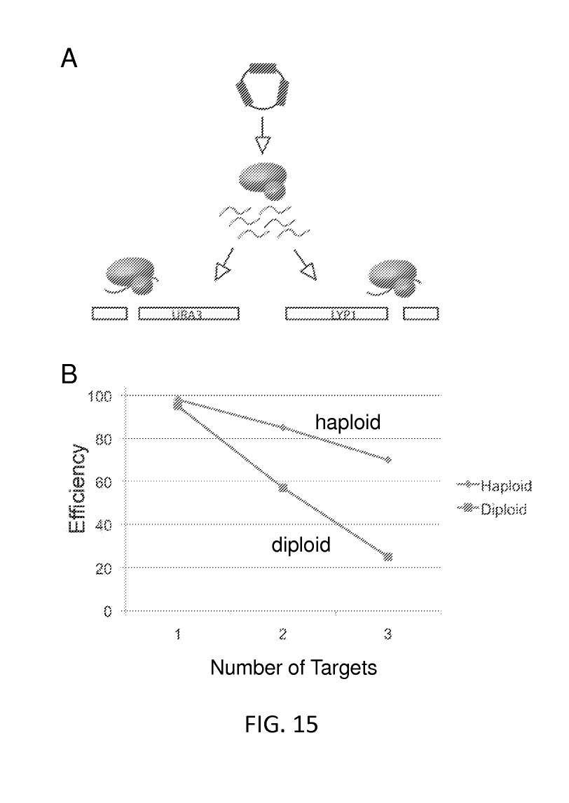

FIG. 15A illustrates an application of multiplex Cas-mediated genome editing. In this example, a plasmid expresses two distinct sgRNAs (e.g., an sgRNA targeting URA3 and an sgRNA targeting LYP1). Targeting efficiency was compared in haploid and diploid yeast cells. As shown in FIG. 15B, targeting efficiency was found to decrease as the number of targeted genetic loci increased, and lower efficiency was consistently observed in diploid cells compared to haploid cells. However, FIG. 15B demonstrates that the system described herein is able to target both diploid and haploid cells for multiplex gene editing. These results demonstrate that the Cas-mediated system described herein is able to facilitate multiplex genome editing, even in diploid cells.

### Example 6

### High-Throughput Protein Engineering Facilitated by Ribozyme-sgRNAs

This Example demonstrates that the methods described in the previous Examples may be used to engineer new functionalities in yeast cells by inserting heterologous enzymes into a yeast genome.

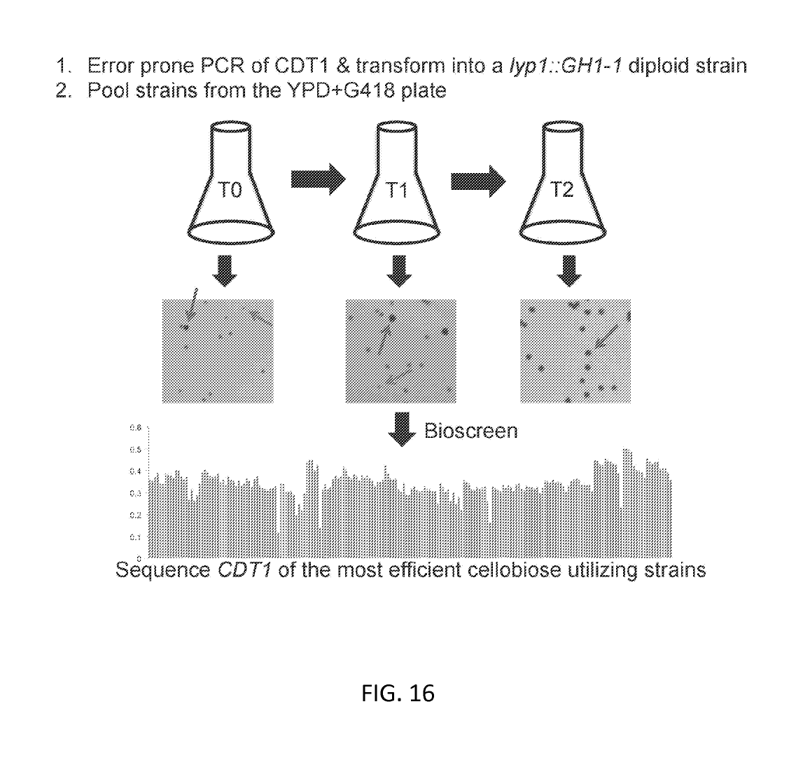

An overview of an exemplary method for using genome editing as described herein is provided in FIG. 16. To select for improved cellobiose utilizing strains, error-prone PCR was used to amplify the cdt-1 gene from N. crassa, and the resulting library of mutated cdt-1 alleles was transformed into a yeast strain (S288C) with a previously integrated β-glucosidase gh1-1 gene. Transformants were grown in liquid medium containing cellobiose as the sole carbon source for two days and plated onto agar containing cellobiose as the sole carbon source. This liquid culture step eliminates the mutant cells with decreased cellobiose utilization phenotypes from the pool and enriches the improved cellobiose utilizing strains on the agar plate. Individual colonies from the cellobiose plates were picked and grown in a 96 well format to compare relative fitness in cellobiose medium compared to wild type.

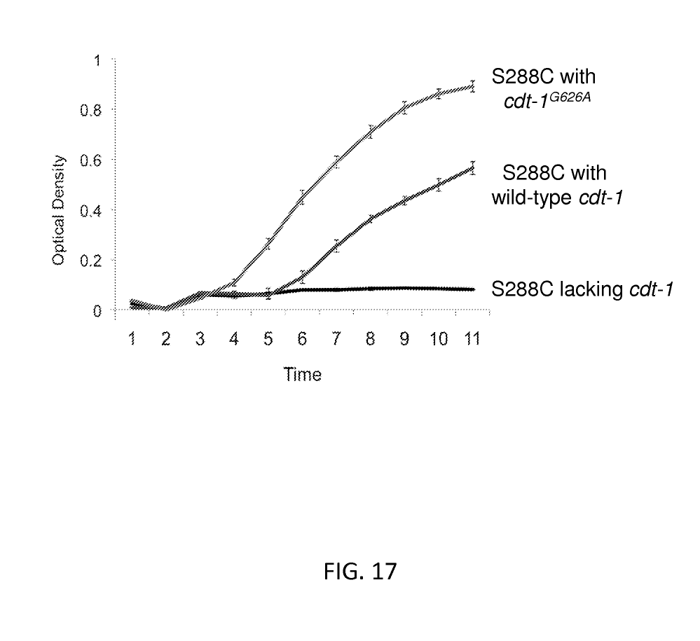

As shown in FIG. 17, a strain (CDT-1^{G626A}) was identified with enhanced cellobiose utilization capacity, compared to wild type cdt-1. As shown, wild-type S288C yeast cells without cdt-1 are not able to utilize cellobiose. Another strain (CDT-1^{T785A}) was identified with 149% of the cellobiose utilization capacity over wild type cdt-1 (not shown). Thus, the CRISPR-Cas9 system may be used to quickly and cost effectively engineer hypermorphic alleles of genes that lead to proteins with improved enzymatic activity, e.g., transporter activity.

The self-cleaving 5′ ribozyme enables use of any tRNA promoter, increasing the number of sgRNA promoters several fold. Further, discovering tRNAs in other organisms through de novo bioinformatics analysis is very simple due to the high conservation of tRNAs. This makes the ribozyme-sgRNA method of RNA expression a portable and universal method for the expression of small non-coding RNAs. Because many RNA polymerase III promoters and terminators are thought to be highly conserved across fungi, using these to express bacterial Cas9 and sgRNA potentially allows this method to be used to perform genome editing in other, more distantly related fungi that have biotechnological uses.

### Example 7

### CRISPR-Cas9 System in Kluyveromyces marxianus

This example demonstrates the use of the CRISPR-Cas9 gene-editing system in another fungal cell type, Kluyveromyces marxianus.

Key regulatory elements were substituted in the original pCAS S. cerevisiae plasmid with their respective sequences isolated from K. marxianus. Those elements were: i) 2 micron origin of replication (KmARS), ii) Cas9-driving promoter (KmpRNR2), iii) Cas9 terminator (KmCYCt) and iv) URA3 20-nucleotide guide RNA (kmURA3). The same transformation protocol as for S. cerevisiae was used except that the repair DNA called “barcode” was not added. K. marxianus prefers a DNA repair pathway that involves non-homologous-end-joining rather than homology-directed repair.

A working CRISPR-Cas9 system should cause a double strand break on the targeted site, then the endogenous DNA repair machinery should repair the break either perfectly or introduce/remove single nucleotides in the vicinity of the damage. The function of this system can be assessed by sequencing the targeted site in Cas9-transformant colonies. The presence of nucleotide insertion or deletions demonstrates that the system is functional.

The transformation efficiency of K. marxianus was similar to S. cerevisiae. Editing efficiency was also high. Three different regions of 3 different loci were targeted: URA3, KU70 and KU80. Transformants were genotyped by randomly picking colonies, sequencing the targeted region and checking for insertions and deletions (INDELs) near the Cas9 restriction site. The URA3 locus showed an editing efficiency of 96% (29 INDELs-bearing colonies out of 30 sequenced). The KU70 locus showed an editing efficiency of 68% (49 INDELs-bearing colonies out of 72 sequenced). The KU80 locus showed an editing efficiency of 70% (36 INDELs-bearing colonies out of 51 sequenced). It is already known that the chromatin topology, and therefore the DNA accessibility at a particular region, is a major factor affecting genome-editing techniques. We believe that this is the reason why editing efficiency varies significantly in different loci.

To assess the efficiency of double-strand-break repair by homology-directed pathways in K. marxianus, the repair fragment (previously referred to as “barcode”) was added to the transformation protocol. The fragment consists of a drug (nourseothricin) resistance cassette flanked by varying sizes of homology arms. The efficiency of integration was very low (on the order of 1%). Although several arm sizes were tested spanning from 40 bp to lkb, no increase in efficiency was found. Although inefficient, the integration due to homology-directed pathways in K. marxianus is highly precise, resulting in on-target integration in more than 80% of the colonies screened, a number never before reported in K. marxianus.

1. An expression vector comprising nucleic acid encoding
an RNA polymerase III promoter;
a ribozyme;
CRISPR-Cas 9 single guide RNA; and
an RNA Polymerase III terminator,
wherein the ribozyme is 5′ to the CRISPR-Cas9 single guide RNA.

2. The expression vector of claim 1, wherein the vector further comprises nucleic acid encoding a Cas9 protein.

3. The expression vector of claim 1, wherein the CRISPR-Cas9 single guide RNA comprises a 20 nucleotide target sequence and a sgRNA (+85) tail.

4. The expression vector of claim 1, wherein the RNA polymerase III promoter is a tRNA.

5. The expression vector of claim 4, wherein the tRNA is a tyrosine tRNA.

6. The expression vector of claim 1, wherein the RNA polymerase III promoter is a non-tRNA promoter.

7. The expression vector of claim 6, wherein the non-tRNA promoter is SNR52.

8. The expression vector of claim 1, wherein the ribozyme is self-cleaving.

9. The expression vector of claim 1, wherein the ribozyme is active between 30° C. and 37° C.

10. The expression vector of claim 1, wherein the ribozyme is a hepatitis delta ribozyme.

11. The expression vector of claim 1, wherein the vector comprises more than one CRISPR-Cas 9 single guide RNA.

12. The ribonucleic acid encoded by the expression vector of claim 1.

13. A fungal cell comprising the expression vector of claim 1.

14. The fungal cell of claim 13, wherein the cell is an industrial strain.

15. The fungal cell of claim 13, wherein the cell is polyploid.

16. The fungal cell of claim 15, wherein the cell is diploid.

17. The fungal cell of claim 13, wherein the cell is a filamentous fungal cell.

18. The fungal cell of claim 13, wherein the cell is a yeast cell.

19. The fungal cell of claim 18, wherein the yeast cell is selected from the group consisting of Saccharomyces cerevisiae, Kluyveromyces marxianus, and Issatchenkia orientalis.

20. A method for engineering a fungal genome, comprising
introducing an expression vector of claim 1 and an expression vector encoding a Cas9 protein into a fungal cell; and
culturing the cell under conditions suitable for expression.

21. A method for engineering a fungal genome, comprising
introducing an expression vector of claim 2 into a fungal cell; and
culturing the cells under conditions suitable for expression.

22. The method of claim 20, further comprising introducing a nucleic acid encoding a gene of interest.

23. The method of claim 22, wherein the gene of interest is a cellodextrin transporter.

24. The method of claim 22, wherein the gene of interest is encoded by more than one polynucleotide.

25. The method of claim 22, wherein the gene of interest is generated by error-prone PCR.
